(19) United States
(12) Patent Application Publication
(10) Pub. No.: US 2024/0352029 A1
(43) Pub. Date: Oct. 24, 2024

(54) CDK Inhibitors And Their Use As Pharmaceuticals

(71) Applicant: Prelude Therapeutics Incorporated, Wilmington, DE (US)

(72) Inventors: Xiaowei Wu, Wilmington, DE (US); Andrew Combs, Kennett Square, PA (US)

(21) Appl. No.: 18/635,202

(22) Filed: Apr. 15, 2024

Related U.S. Application Data
(60) Provisional application No. 63/496,111, filed on Apr. 14, 2023.

(51) Int. Cl. C07D 495/04; A61K 31/4196; A61K 31/506; A61K 31/565; A61P 35/00

(52) U.S. Cl. CPC C07D 495/04; A61K 31/4196; A61K 31/506; A61K 31/565; A61P 35/00

(57) ABSTRACT

The disclosure is directed to compounds of Formula I

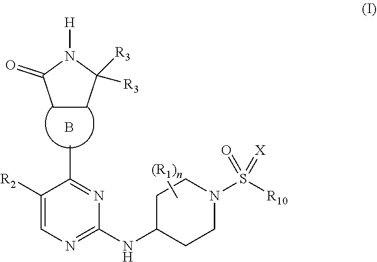

pharmaceutical compositions comprising compounds of Formula I, as well as methods of their use and preparation, are also described.

### CROSS-REFERENCE TO RELATED APPLICATIONS

The present claims benefit of U.S. Provisional application No. 63/496,111, filed Apr. 14, 2023, the entire contents of each of which are incorporated herein by this reference.

### TECHNICAL FIELD

The disclosure is directed to CDK inhibitors and methods of their use.

### BACKGROUND

Cyclin-dependent kinases (CDKs) are a family of conserved serine/threonine kinases that play critical roles in cell cycle and gene transcription regulation (Malumbres 2014). Among the cell cycle CDK subfamily, CDK4 and CDK6 are the master regulators that control entry of cells from the first gap phase (G1) to the DNA synthesis phase (S). During this process, cyclin D protein levels increase, complex with CDK4/6 and activate their kinase activities. Activated CDK4/6 complexes phosphorylate retinoblastoma protein (RB1) and other RB1-like proteins, reduce their binding affinities and release RB1-containing transcription repressor complexes from E2F transcription factors, resulting in activation of E2F controlled cell cycle genes and progression of cell cycle (Lapenna and Giordano 2009, Asghar, Witkiewicz et al. 2015).

Additional small molecule CDK2/4/6 inhibitors are needed.

### SUMMARY OF THE INVENTION

The disclosure is directed to compounds of Formula I:

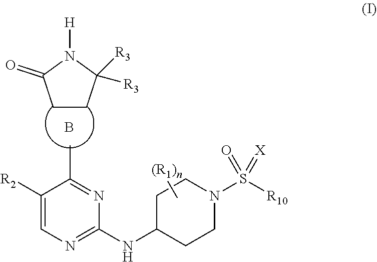

or a pharmaceutically acceptable salt or solvate or N-oxide thereof, wherein ring B is a 5-membered heteroaryl selected from:

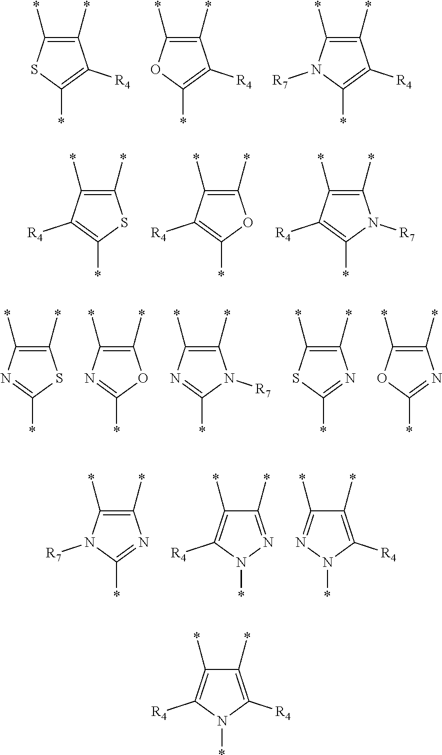

n is 0, 1, 2, 3, 4, 5, 6, 7, 8 or 9; each R₁, when present, is independently H, D, halogen, —OH, —CN, —NO₂, —C₁-C₆ alkyl, C₁₋₆alkoxide, —C₂-C₆ alkenyl, —C₂-C₆ alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or two R₁ groups together with the atoms to which they are both attached form a carbocyclic or heterocyclic group;
R₂ is H, D, halogen, C₁-C₈ alkoxide, C₁-C₈ alkyl, haloalkoxide, C₁-C₈ haloalkyl, SF₅, or CN, wherein the C₁₋₈ alkyl is optionally substituted with D, halogen, —OH, —CN, or cycloalkyl;
each R₃ is independently H, D, halogen, —OH, —CN, —NO₂, —C₁-C₆ alkyl, —C₂-C₆ alkenyl, —C₂-C₆alkynyl, C₀-C₁ alk-aryl, C₀-C₁ alk-heteroaryl, cycloalkyl, cycloalkenyl, heterocycloalkyl, heterocycloalkenyl, —OR^{a}, —SR^{b}, —NR^{c}R^{d}, —NR^{a}R^{c}, —C(O)R^{b}, —OC(O)R^{b}, —C(O)OR^{b}, —C(O)NR^{c}R^{d}, or —B(OR^{d})(OR^{c}); wherein the —C₁-C₆ alkyl, —C₂-C₆ alkenyl, —C₂-C₆ alkynyl, C₀-C₁ alk-aryl, C₀-C₁ alk- heteroaryl, cycloalkyl, cycloalkenyl, heterocycloalkyl and heterocycloalkenyl are optionally substituted with at least one of D, halogen, —OH, —CN, an amine, cycloalkyl, or heterocycloalkyl;
each R₄ is independently H, D, halogen, C₁-C₈ alkoxide, C₁-C₈ alkyl, C₃-C₈ cycloalkyl, C₄-C₈ heterocyclyl, —C(O)NR^{c}R^{d}, SF₅ or CN, wherein the C₁₋₈ alkyl, C₃-C₈ cycloalkyl, or C₄-C₈ heterocyclyl are optionally substituted with D, halogen, —OH, —CN, or cycloalkyl;
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, SiR^{b}₃, —C₁-C₁₀ alkyl, —C₂-C₁₀ alkenyl, —C₂-C₁₀ alkynyl, C₀-C₁ alk-aryl, cycloalkyl, cycloalkenyl, C₀-C₁ alk-heteroaryl, heterocycloalkyl, or heterocycloalkenyl;
each R^{b} is independently H, D, —C₁-C₆ alkyl, —C₂-C₆ alkenyl, —C₂-C₆ alkynyl, C₀-C₁ alk-aryl, cycloalkyl, cycloalkenyl, C₀-C₁ alk-heteroaryl, heterocycloalkyl, or heterocycloalkenyl;
each R^{c} is independently H, D, —C₁-C₁₀ alkyl, —C₂-C₆ alkenyl, —C₂-C₆ alkynyl, —OC₁-C₆ alkyl, —O-cycloalkyl, aryl, C₁ alk-aryl, heteroaryl, cycloalkyl, cycloalkenyl, C₁ alk-heteroaryl, heterocycloalkyl, or heterocycloalkenyl;
each R^{d} is independently H, D, —C₁-C₁₀ alkyl, —C₂-C₆ alkenyl, —C₂-C₆ alkynyl, —OC₁-C₆alkyl, —O-cycloalkyl, aryl, C₁alk-aryl, heteroaryl, cycloalkyl, cycloalkenyl, C₁alk-heteroaryl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group;
R₇ is H, D, OR^{a}, C₁₋₄alkyl, wherein the C₁₋₄alkyl are optionally substituted with at least one of D, halogen, —OH, —CN, an amine, cycloalkyl, or heterocycloalkyl;
R₁₀ is H, D, —NR^{c}R^{d}, —NR^{a}R^{c}, C₁₋₆alkyl, C₃₋₇cycloalkyl, C₄₋₇heterocycloalkyl, C₃₋₇ cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇cycloalkylalkyl, C₄₋₇ heterocycloalkylalkyl, aryl, heteroaryl, arylalkyl and heteroarylalkyl are optionally substituted by 1-6 R groups selected from H, D, halogen, —OH, —CN, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or R¹ and R¹⁰ together with the atoms to which they are both attached form a heterocyclic group which are optionally substituted with D, halogen, —OH, —CN, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

Stereoisomers of the compounds of Formula I, as well as the pharmaceutical salts, solvates, and N-oxides thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

At various places in the present specification, substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. Alkyl groups of the disclosure can be unsubstituted or substituted. In those embodiments wherein the alkyl group is substituted, the alkyl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “alkoxide” refers to the conjugate base of an alcohol and includes an organic group bonded to a negatively charged oxygen atom.

The term “halo” or halogen, refers to chloro, fluoro, bromo, or iodo.

The term “haloalkyl” refers to any alkyl radical having one or more hydrogen atoms replaced by a halogen atom.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Cycloalkyl groups of the disclosure include monocyclic groups, as well as multicyclic groups such as bicyclic and tricyclic groups. In those embodiments having at least one multicyclic cycloalkyl group, the cyclic groups can share one common atom (i.e., spirocyclic). In other embodiments having at least one multicyclic cycloalkyl group, the cyclic groups share two common atoms. Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), spiro[3.3]heptanyl, bicyclo[3.3.0]octanyl, and the like. Cycloalkyl groups of the disclosure can be unsubstituted or substituted. In those embodiments wherein the cycloalkyl group is substituted, the cycloalkyl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “cycloalkenyl” refer to cyclic, non-aromatic hydrocarbon groups having from 3 to 10 carbon atoms (“C₃-C₁₀”), preferably from 3 to 6 carbon atoms (“C₃-C₆”) and containing at least one carbon-carbon double bond. For example, cycloalkenyl groups include, but are not limited to cyclopropenyl, cyclobutenyl, and the like.

The term “heterocycloalkyl” when used alone or as part of a substituent group refers to any three to ten membered monocyclic or bicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic heterocycloalkyl group, the cyclic groups can share one common atom (i.e., spirocyclic). In other embodiments having at least one multicyclic heterocycloalkyl group, the cyclic groups share two common atoms. The term —C₃-C₆ heterocycloalkyl refers to a heterocycloalkyl group having between three and six carbon ring atoms. The term —C₃-C₁₀ heterocycloalkyl refers to a heterocycloalkyl group having between three and 10 ring atoms. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, azepanyl, diazepanyl, oxepanyl, dioxepanyl, azocanyl diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, and the like. Heteroycloalkyl groups of the disclosure can be unsubstituted or substituted. In those embodiments wherein the heterocycloalkyl group is substituted, the heterocycloalkyl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “heterocycloalkenyl” when used alone or as part of a substituent group refers to any three to ten membered monocyclic or bicyclic, partially saturated ring structure containing at least one heteroatom selected from the group consisting of O, N and S. Heterocycloalkenyl groups of the disclosure include monocyclic groups, as well as multicyclic groups such as bicyclic and tricyclic groups. In those embodiments having at least one multicyclic heterocycloalkyenyl group, the cyclic groups can share one common atom (i.e., spirocyclic). In other embodiments having at least one multicyclic heterocycloalkenyl group, the cyclic groups share two common atoms. The term —C₃-C₆ heterocycloalkenyl refers to a heterocycloalkenyl group having between three and six carbon atoms. The term —C₃-C₁₀ heterocycloalkenyl refers to a heterocycloalkenyl group having between three and ten ring atoms. The heterocycloalkenyl group may be attached at any heteroatom or carbon atom of the ring such that the result is a stable structure. Heteroycloalkenyl groups of the disclosure can be unsubstituted or substituted. In those embodiments wherein the heterocycloalkenyl group is substituted, the heterocycloalkenyl group can be substituted with 1, 2, or 3 substituents independently selected from D, —OH, —CN, amino, halo, oxo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “heteroaryl” when used alone or as part of a substituent group refers to a mono- or bicyclic-aromatic ring structure including carbon atoms as well as up to five heteroatoms selected from nitrogen, oxygen, and sulfur. Heteroaryl rings can include a total of 5, 6, 7, 8, 9, or 10 ring atoms. The term —C₅-C₁₀ heteroaryl refers to a heteroaryl group containing five to ten ring atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. Heteroaryl groups of the disclosure can be unsubstituted or substituted. In those embodiments wherein the heteroaryl group is substituted, the heteroaryl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “aryl” when used alone or as part of a substituent group refers to a mono- or bicyclic-aromatic carbon ring structure. Aryl rings can include a total of 6, 7, 8, 9, or 10 ring atoms. Examples of aryl groups include but are not limited to, phenyl, napthyl, and the like. Aryl groups of the disclosure can be unsubstituted or substituted. In those embodiments wherein the aryl group is substituted, the aryl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “alkenyl” refers to C₂-C₁₂ alkyl group that contains at least one carbon-carbon double bond. In some embodiments, the alkenyl group is optionally substituted. In some embodiments, the alkenyl group is a C₂-C₆ alkenyl.

The term “alkynyl” refers to C₂-C₁₂ alkyl group that contains at least one carbon-carbon triple bond. In some embodiments, the alkenyl group is optionally substituted. In some embodiments, the alkynyl group is a C₂-C₆ alkynyl.

As used herein, “alkoxy” refers to an —O-alkyl group. Example alkoxy groups include methoxy, ethoxy, propoxy (e.g., n-propoxy and isopropoxy), t-butoxy, and the like.

As used herein, “hydroxylalkyl” refers to an alkyl group substituted by OH.

When a range of carbon atoms is used herein, for example, C₁-C₆, all ranges, as well as individual numbers of carbon atoms are encompassed, for example, “C₁₋₃” includes C₁₋₃, C₁₋₂, C₂₋₃, C₁, C₂, and C₃. The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond.

The term “C₀-C₆alk” when used alone or as part of a substituent group refers to an aliphatic linker having 0, 1, 2, 3, 4, 5 or 6 carbon atoms. The term “—C₁alk-”, for example, refers to a —CH₂—. The term “—C₀alk-” refers to a bond.

Moieties of the disclosure, for example, —C₁-C₆alkyl, —C₁-C₁₀ alkyl, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalky, are optionally substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Geometric isomers of the compounds of the present invention are described and may be isolated as a mixture of isomers or as separated isomeric forms.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

Compounds of the invention may also include tautomeric forms. All tautomeric forms are encompassed.

In some embodiments, the compounds of the present invention may exist as rotational isomers. In some embodiments, the compounds of the present invention exist as mixtures of rotational isomers in any proportion. In other embodiments, the compounds of the present invention exist as particular rotational isomers, substantially free of other rotational isomers.

In some embodiments, the compounds of the invention, and salts thereof, are substantially isolated. By “substantially isolated” is meant that the compound is at least partially or substantially separated from the environment in which was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes mammals, and in particular, humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as its subgenera, which expression includes the stereoisomers (e.g., entaniomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

Compounds of the invention can also include all isotopes of atoms occurring in the intermediates or final compounds. Isotopes include those atoms having the same atomic number but different mass numbers. For example, isotopes of hydrogen include tritium and deuterium.

The disclosure is directed to compounds of Formula I:

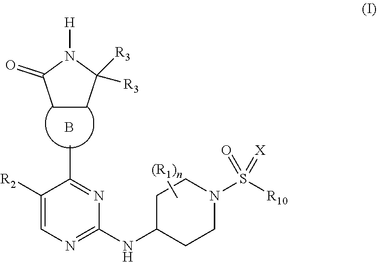

or a pharmaceutically acceptable salt or solvate or N-oxide thereof, wherein
ring B is a 5-membered heteroaryl selected from:

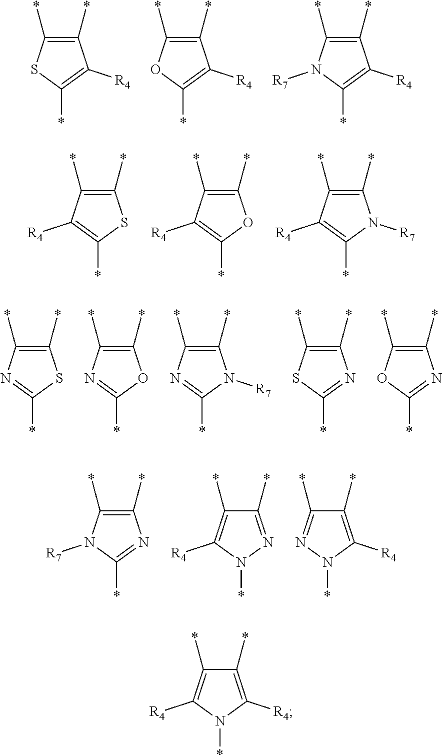

n is 0, 1, 2, 3, 4, 5, 6, 7, 8 or 9;
each R₁, when present, is independently H, D, halogen, —OH, —CN, —NO₂, —C₁-C₆ alkyl, C₁₋₆alkoxide, —C₂-C₆ alkenyl, —C₂-C₆ alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or two R₁ groups together with the atoms to which they are both attached form a carbocyclic or heterocyclic group;
R₂ is H, D, halogen, C₁-C₈ alkoxide, C₁-C₈ alkyl, haloalkoxide, C₁-C₈ haloalkyl, SF₅, or CN, wherein the C₁₋₈ alkyl is optionally substituted with D, halogen, —OH, —CN, or cycloalkyl;
each R₃ is independently H, D, halogen, —OH, —CN, —NO₂, —C₁-C₆ alkyl, —C₂-C₆ alkenyl, —C₂-C₆alkynyl, C₀-C₁ alk-aryl, C₀-C₁ alk-heteroaryl, cycloalkyl, cycloalkenyl, heterocycloalkyl, heterocycloalkenyl, —OR^{a}, —SR^{b}, —NR^{c}R^{d}, —NR^{a}R^{c}, —C(O)R^{b}, —OC(O)R^{b}, —C(O)OR^{b}, —C(O)NR^{c}R^{d}, or —B(OR^{d})(OR^{c}); wherein the —C₁-C₆ alkyl, —C₂-C₆ alkenyl, —C₂-C₆ alkynyl, C₀-C₁ alk-aryl, C₀-C₁ alk- heteroaryl, cycloalkyl, cycloalkenyl, heterocycloalkyl and heterocycloalkenyl are optionally substituted with at least one of D, halogen, —OH, —CN, an amine, cycloalkyl, or heterocycloalkyl;
each R₄ is independently H, D, halogen, C₁-C₈ alkoxide, C₁-C₈ alkyl, C₃-C₈ cycloalkyl, C₄-C₈ heterocyclyl, —C(O)NR^{c}R^{d}, SF₅ or CN, wherein the C₁₋₈ alkyl, C₃-C₈ cycloalkyl, or C₄-C₈ heterocyclyl are optionally substituted with D, halogen, —OH, —CN, or cycloalkyl;
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, SiR^{b}₃, —C₁-C₁₀ alkyl, —C₂-C₁₀ alkenyl, —C₂-C₁₀ alkynyl, C₀-C₁ alk-aryl, cycloalkyl, cycloalkenyl, C₀-C₁ alk-heteroaryl, heterocycloalkyl, or heterocycloalkenyl;
each R^{b} is independently H, D, —C₁-C₆ alkyl, —C₂-C₆ alkenyl, —C₂-C₆ alkynyl, C₀-C₁ alk-aryl, cycloalkyl, cycloalkenyl, C₀-C₁ alk-heteroaryl, heterocycloalkyl, or heterocycloalkenyl;
each R^{c} is independently H, D, —C₁-C₁₀ alkyl, —C₂-C₆ alkenyl, —C₂-C₆ alkynyl, —OC₁-C₆ alkyl, —O-cycloalkyl, aryl, C₁ alk-aryl, heteroaryl, cycloalkyl, cycloalkenyl, C₁ alk-heteroaryl, heterocycloalkyl, or heterocycloalkenyl;
each R^{d} is independently H, D, —C₁-C₁₀ alkyl, —C₂-C₆ alkenyl, —C₂-C₆ alkynyl, —OC₁-C₆alkyl, —O-cycloalkyl, aryl, C₁alk-aryl, heteroaryl, cycloalkyl, cycloalkenyl, C₁alk-heteroaryl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group;
R₇ is H, D, OR^{a}, C₁₋₄alkyl, wherein the C₁₋₄alkyl are optionally substituted with at least one of D, halogen, —OH, —CN, an amine, cycloalkyl, or heterocycloalkyl;
R₁₀ is H, D, —NR^{c}R^{d}, —NR^{a}R^{c}, C₁₋₆alkyl, C₃₋₇cycloalkyl, C₄₋₇heterocycloalkyl, C₃₋₇ cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇cycloalkylalkyl, C₄₋₇ heterocycloalkylalkyl, aryl, heteroaryl, arylalkyl and heteroarylalkyl are optionally substituted by 1-6 R groups selected from H, D, halogen, —OH, —CN, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or R¹ and R¹⁰ together with the atoms to which they are both attached form a heterocyclic group which are optionally substituted with D, halogen, —OH, —CN, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, ring B in Formula (I) is a 5-membered heteroaryl selected from:

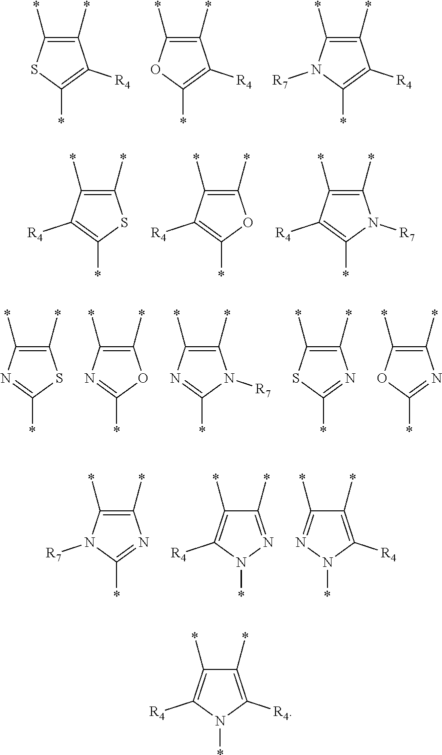

In some embodiments, ring B is

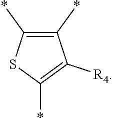

In other embodiments, ring B is

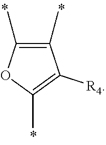

In yet other embodiments, ring B is

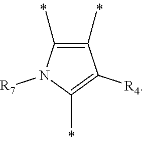

In yet other embodiments, ring B is

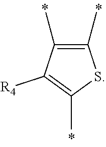

In yet other embodiments, ring B is

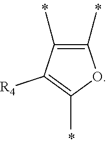

In yet other embodiments, ring B is

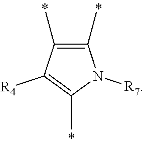

In yet other embodiments, ring B is

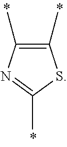

In yet other embodiments, ring B is

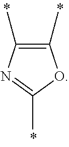

In yet other embodiments, ring B is

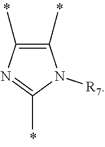

In yet other embodiments, ring B is

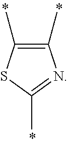

In yet other embodiments, ring B is

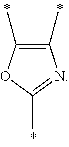

In yet other embodiments, ring B is

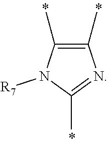

In yet other embodiments, ring B is

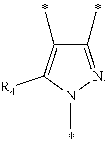

In yet other embodiments, ring B is

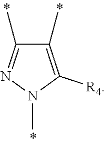

In yet other embodiments, ring B is

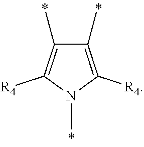

In some embodiments, each R₁, when present, in Formula I is independently H, D, halogen, —OH, —CN, —NO₂, —C₁-C₆alkyl, C₁₋₆alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or two R¹ groups together with the atoms to which they are both attached form a carbocyclic or a heterocyclic group.

In some embodiments, R₁ is independently H. In some embodiments, R₁ is independently D. In some embodiments, R₁ is independently halogen. In some embodiments, R₁ is independently —OH. In some embodiments, R₁ is independently —CN. In some embodiments, R₁ is independently NO₂. In some embodiments, R₁ is independently —C₁-C₆alkyl. In some embodiments, R₁ is independently C₁₋₆alkoxide. In some embodiments, R₁ is independently —C₂-C₆alkenyl. In some embodiments, R₁ is independently —C₂-C₆alkynyl. In some embodiments, R₁ is independently aryl. In some embodiments, R₁ is independently heteroaryl. In some embodiments, R₁ is independently cycloalkyl. In some embodiments, R₁ is independently cycloalkenyl. In some embodiments, R₁ is independently heterocycloalkenyl. In some embodiments, R₁ is independently —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}), —S(O)₂R^{b} and the like.

In other embodiments, wherein at least one R₁ is H. In other embodiments, wherein at least one R₁ is —C₁-C₆ alkyl. In some embodiments, wherein at least one R₁ is methyl. In yet other embodiments, wherein at least one R₁ is halogen. In yet other embodiments, wherein at least one R₁ is F.

In yet other embodiments, two R₁ groups together with the atoms to which they are both attached form a carbocyclic or a heterocyclic group.

In some embodiments, n in Formula (I) is 0, 1, 2, 3, 4, 5, 6, 7, 8 or 9. In some embodiments, n is 0. In some embodiments, n is 1. In some embodiments, n is 2. In other embodiments, n is 3. In other embodiments, n is 4. In other embodiments, n is 5. In other embodiments, n is 6. In other embodiments, n is 7. In other embodiments, n is 8. In other embodiments, n is 9.

In some embodiments, R₂ in Formula I is H, D, halogen, C₁-C₈ alkoxide, C₁-C₈ alkyl, haloalkoxide, C₁-C₈ haloalkyl, SF₅, or CN, wherein the C₁₋₈alkyl is optionally substituted with D, halogen, —OH, —CN, or cycloalkyl. In some embodiments, R₂ is H. In some embodiments, R₂ is D. In some embodiments, R₂ is halogen. In some embodiments, R₂ is C₁-C₈ alkoxide. In other embodiments, R₂ is C₁-C₈ alkyl. In other embodiments, R₂ is haloalkoxide. In other embodiments, R₂ is C₁-C₈ haloalkyl. In yet other embodiments, R₂ is SF₅. In yet other embodiments, R₂ is —CN. In yet other embodiments, the C₁₋₈ alkyl group of R₂ is substituted with D, halogen, —OH, —CN, or cycloalkyl.

In some embodiments, R₂ in Formula I is halogen. In some embodiments, R₂ in Formula I is F. In some embodiments, R₂ in Formula I is C₁-C₈ haloalkyl. In some embodiments, R₂ in Formula I is CF₃.

In some embodiments, each R₃ in Formula I is independently H, D, halogen, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, heterocycloalkenyl, —OR^{a}, —SR^{b}, —NR^{c}R^{d}, —NR^{a}R^{c}, —C(O)R^{b}, —OC(O)R^{b}, —C(O)OR^{b}, —C(O)NR^{c}R^{d}, or —B(OR^{d})(OR^{c}); wherein the —C₁-C₆ alkyl, —C₂-C₆ alkenyl, —C₂-C₆alkynyl, C₀-C₁ alk-aryl, C₀-C₁ alk-heteroaryl, cycloalkyl, cycloalkenyl, heterocycloalkyl and heterocycloalkenyl are optionally substituted with at least one of D, halogen, —OH, —CN, an amine, cycloalkyl, or heterocycloalkyl.

In some embodiments, R₃ is independently H. In some embodiments, R₃ is independently D. In some embodiments, R₃ is independently halogen. In some embodiments, R₃ is independently —OH. In some embodiments, R₃ is independently —CN. In some embodiments, R₃ is independently —NO₂. In other embodiments, R₃ is independently —C₁-C₆alkyl. In other embodiments, R₃ is independently —C₂-C₆alkenyl. In other embodiments, R₃ is independently —C₂-C₆alkynyl. In other embodiments, R₃ is independently C₀-C₁alk-aryl. In other embodiments, R₃ is independently C₀-C₁alk-heteroaryl. In other embodiments, R₃ is independently cycloalkyl. In other embodiments, R₃ is independently cycloalkenyl. In other embodiments, R₃ is independently heterocycloalkyl. In other embodiments, R₃ is independently heterocycloalkenyl. In yet other embodiments, R₃ is independently —OR^{a}. In yet other embodiments, R₃ is independently —SR^{b}. In yet other embodiments, R₃ is independently —NR^{c}R^{d}. In yet other embodiments, R₃ is independently —NR^{a}R^{c}. In yet other embodiments, R₃ is independently —C(O)R^{b}. In yet other embodiments, R₃ is independently —OC(O)R^{b}. In yet other embodiments, R₃ is independently —C(O)NR^{c}R^{d}. In yet other embodiments, R₃ is independently B(OR^{d})(OR^{c}).

In yet other embodiments, the —C₁-C₆ alkyl, —C₂-C₆ alkenyl, —C₂-C₆ alkynyl, C₀-C₁ alk-aryl, C₀-C₁ alk-heteroaryl, cycloalkyl, cycloalkenyl, heterocycloalkyl and heterocycloalkenyl are optionally substituted with at least one of D, halogen, —OH, —CN, an amine, cycloalkyl, or heterocycloalkyl.

In some embodiments, R₃ at least one R₃ is H. In some embodiments, each R₃ is H. In some embodiments, at least one R₃ is —C₁-C₆ alkyl. In other embodiments, each R₃ is —C₁-C₆ alkyl. In other embodiments, at least one R₃ is methyl. In other embodiments, each R₃ is methyl.

In some embodiments, each R₄ in Formula I is independently H, D, halogen, C₁-C₈ alkoxide, C₁-C₈ alkyl, C₃-C₈ cycloalkyl, C₄-C₈ heterocyclyl, —C(O)NR^{c}R^{d}, SF₅ or CN, wherein the C₁₋₈alkyl, C₃-C₈ cycloalkyl, or C₄-C₈ heterocyclyl are optionally substituted with D, halogen, —OH, —CN, or cycloalkyl.

In some embodiments, at least one R₄ in Formula I is H. In some embodiments, at least one R₄ in Formula I is D. In some embodiments, at least one R₄ in Formula I is halogen. In some embodiments, at least one R₄ in Formula I is —C₁-C₈ alkoxide. In other embodiments, at least one R₄ in Formula I is —C₁-C₈ alkyl. In other embodiments, at least one R₄ is C₃-C₈ cycloalkyl. In other embodiments, at least one R₄ in Formula I is C₄-C₈ heterocyclyl. In yet other embodiments, at least one R₄ in Formula I is —C(O)NR^{c}R^{d}. In yet other embodiments, at least one R₄ in Formula I is SF₅. In yet other embodiments, at least one R₄ in Formula I is —CN. In yet other embodiments, the C₁₋₈alkyl, C₃-C₈ cycloalkyl, or C₄-C₈ heterocyclyl of R₄ in Formula I is substituted with D, halogen, —OH, —CN, or cycloalkyl.

In some embodiments, at least one R₄ is H. In some embodiments, at least one R₄ is —C₁-C₆ alkyl. In some embodiments, at least one R₄ is methyl.

In some embodiments, R₇ in Formula I is H, D, OR^{a}, C₁₋₄alkyl, wherein the C₁₋₄alkyl is optionally substituted with at least one of D, halogen, —OH, —CN, an amine, cycloalkyl, or heterocycloalkyl. In some embodiments, R₇ in Formula I is H. In some embodiments, R₇ in Formula I is D. In other embodiments, R₇ in Formula I is OR^{a}. In other embodiments, R₇ in Formula I is C₁₋₄alkyl. In other embodiments, R₇ in Formula I is methyl. In other embodiments, R₇ in Formula I is ethyl. In yet other embodiments, R₇ in Formula I is propyl. In yet other embodiments, R₇ in Formula I is isopropyl. In yet other embodiments, R₇ in Formula I is butyl. In yet other embodiments, R₇ in Formula I is t-butyl. In some embodiments, the C₁₋₄alkyl of R₇ in Formula I is substituted with at least one of D, halogen, —OH, —CN, an amine, cycloalkyl, or heterocycloalkyl.

In some embodiments, R₁₀ in Formula I is H, D, —NR^{c}R^{d}, —NR^{a}R^{c}, C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇ cycloalkylalkyl, C₄₋₇heterocycloalkylalkyl, aryl, heteroaryl, arylalkyl and heteroarylalkyl are optionally substituted by 1-6 R selected from H, D, halogen, —OH, —CN, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R₁₀ in Formula I is C₁₋₆alkyl. In some embodiments, R₁₀ is methyl. In other embodiments, R₁₀ is ethyl. In other embodiments, R₁₀ is propyl. In yet other embodiments, R₁₀ is isopropyl. In yet other embodiments, R₁₀ is butyl. In yet other embodiments, R₁₀ is t-butyl. In yet other embodiments, R₁₀ is pentyl. In yet other embodiments, R₁₀ is neopentyl. In yet other embodiments, R₁₀ is hexyl.

In some embodiments, R₁ and R₁₀ together with the atoms to which they are both attached form a heterocyclic group which are optionally substituted with D, halogen, —OH, —CN, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, each R^{a} in Formula I is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl.

In some embodiments, R^{a} is independently H. In some embodiments, R^{a} is independently D. In some embodiments, R^{a} is independently —C(O)R^{b}. In some embodiments, R^{a} is independently —C(O)OR^{c}. In some embodiments, R^{a} is independently —C(O)NR^{c}R^{d}. In some embodiments, R^{a} is independently —C(═NR^{b})NR^{b}R^{c}. In some embodiments, R^{a} is independently C(═NOR^{b})NR^{b}R^{c}. In some embodiments, R^{a} is independently —C(═NCN)NR^{b}R^{c}.

In other embodiments, R^{a} is independently P(OR^{c})₂, —P(O)OR^{c}OR^{b}, —S(O)₂R^{b}, —S(O)₂NR^{c}R^{d}, SiR^{b}₃, and the like. In yet other embodiments, R^{a} is independently —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl, and the like.

In some embodiments, each R^{b} in Formula I is independently H, D, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl. In some embodiments, R^{b} is independently H. In some embodiments, R^{b} is independently D. In some embodiments, R^{b} is independently —C₁-C₆ alkyl. In some embodiments, R^{b} is independently —C₂-C₆ alkenyl. In some embodiments, R^{b} is independently —C₂-C₆ alkynyl. In other embodiments, R^{b} is independently C₀-C₁alk-aryl. In other embodiments, R^{b} is independently cycloalkyl. In other embodiments, R^{b} is independently cycloalkenyl. In other embodiments, R^{b} is independently C₀-C₁alk-heteroaryl. In other embodiments, R^{b} is independently heterocycloalkyl. In other embodiments, R^{b} is independently heterocycloalkenyl.

In some embodiments, each R^{c} in Formula I is independently H, D, —C₁-C₆ alkyl, —C₂-C₆ alkenyl, —C₂-C₆ alkynyl, aryl, C₁alk-aryl, cycloalkyl, cycloalkenyl, C₁alk-heteroaryl, heteroaryl, heterocycloalkyl, or heterocycloalkenyl. In some embodiments, R^{c} is independently H. In some embodiments, R^{c} is independently D. In some embodiments, R^{c} is independently —C₁-C₁₀ alkyl. In some embodiments, R^{c} is independently —C₂-C₆ alkenyl. In some embodiments, R^{c} is independently —C₂-C₆ alkynyl. In other embodiments, R^{c} is independently —OC₁-C₆alkyl. In other embodiments, R^{c} is independently —O-cycloalkyl. In other embodiments, R^{c} is independently aryl. In other embodiments, R^{c} is independently C₁alk-aryl. In other embodiments, R^{c} is independently cycloalkyl. In other embodiments, R^{c} is independently cycloalkenyl. In other embodiments, R^{c} is independently C₁alk-heteroaryl. In other embodiments, R^{c} is independently heteroaryl. In other embodiments, R^{c} is independently heterocycloalkyl. In other embodiments, R^{c} is independently heterocycloalkenyl.

In some embodiments, each R^{d} in Formula I is independently H, D, —C₁-C₆ alkyl, —C₂-C₆ alkenyl, —C₂-C₆ alkynyl, aryl, C₁alk-aryl, cycloalkyl, cycloalkenyl, C₁alk-heteroaryl, heteroaryl, heterocycloalkyl, or heterocycloalkenyl. In some embodiments, R^{d} is independently H. In some embodiments, R^{d} is independently D. In some embodiments, R^{d} is independently —C₁-C₁₀ alkyl. In some embodiments, R^{d} is independently —C₂-C₆ alkenyl. In some embodiments, R^{d} is independently —C₂-C₆ alkynyl. In other embodiments, R^{d} is independently —OC₁-C₆alkyl. In other embodiments, R^{d} is independently —O-cycloalkyl. In other embodiments, R^{d} is independently aryl. In other embodiments, R^{d} is independently C₁alk-aryl. In other embodiments, R^{d} is independently cycloalkyl. In other embodiments, R^{c} or R^{d} is independently cycloalkenyl. In other embodiments, R^{d} is independently C₁alk-heteroaryl. In other embodiments, R^{d} is independently heteroaryl. In other embodiments, R^{d} is independently heterocycloalkyl. In other embodiments, R^{d} is independently heterocycloalkenyl.

In yet other embodiments, R^{c} and R^{d}, together with the atoms to which they are both attached, form a monocyclic or multicyclic heterocycloalkyl, or a monocyclic or multicyclic heterocyclo-alkenyl group. In yet other embodiments, R^{c} and R^{d}, together with the atoms to which they are both attached, form a monocyclic heterocycloalkyl. In yet other embodiments, R^{c} and R^{d}, together with the atoms to which they are both attached, form a multicyclic heterocycloalkyl. In yet other embodiments, R^{c} and R^{d}, together with the atoms to which they are both attached, form a monocyclic heterocyclo-alkenyl group. In yet other embodiments, R^{c} and R^{d}, together with the atoms to which they are both attached, form a multicyclic heterocyclo-alkenyl group.

In some embodiments, the compounds of Formula (I) are the pharmaceutically acceptable salts. In some embodiments, the compounds of Formula (I) are solvates. In some embodiments, the compounds of Formula (I) are N-oxides of the compounds of Formula (I).

In some embodiments, the compounds of Formula (I) are represented by compounds of Formula II:

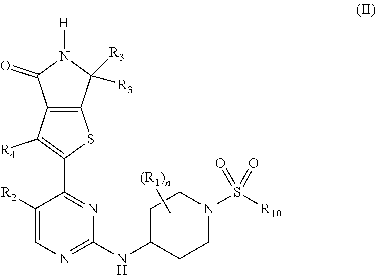

or a pharmaceutically acceptable salt or solvate or N-oxide thereof; wherein each (R₁)ₙ, R₂, R₃, R₄ and R₁₀ are defined with respect to Formula (I).

In some embodiments, the compounds of Formula (I) are represented by compounds of Formula III:

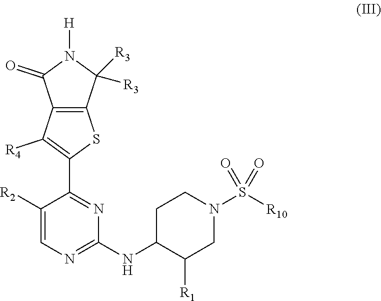

or a pharmaceutically acceptable salt or solvate or N-oxide thereof; wherein each R₁, R₂, R₃, R₄ and R₁₀ are defined with respect to Formula (I).

In some embodiments, the compounds of Formula (I) are represented by compounds of Formula IV:

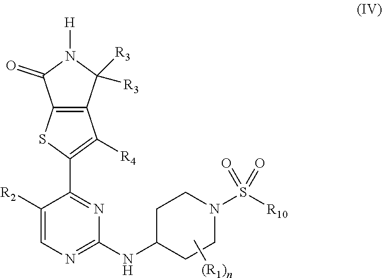

or a pharmaceutically acceptable salt or solvate or N-oxide thereof; wherein each (R₁)ₙ, R₂, R₃, R₄ and R₁₀ are defined with respect to Formula (I).

In some embodiments, the compounds of Formula (I) are represented by compounds of Formula V:

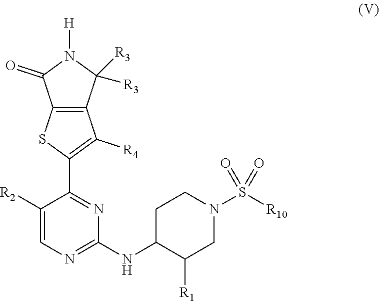

or a pharmaceutically acceptable salt or solvate or N-oxide thereof; wherein each R₁, R₂, R₃, R₄ and R₁₀ are defined with respect to Formula (I).

In yet further embodiments, the compounds of Formula (I) are:
2-(5-Fluoro-2-(((3R,4S)-3-methyl-1-(methylsulfonyl)piperidin-4-yl)amino)pyrimidin-4-yl)-3,6,6-trimethyl-5,6-dihydro-4H-thieno[2,3-c]pyrrol-4-one;
2-(5-fluoro-2-(((3R,4S)-3-fluoro-1-(methylsulfonyl)piperidin-4-yl)amino)pyrimidin-4-yl)-3,6,6-trimethyl-5,6-dihydro-4H-thieno[2,3-c]pyrrol-4-one;
2-(5-fluoro-2-((1-(methylsulfonyl)piperidin-4-yl)amino)pyrimidin-4-yl)-3,6,6-trimethyl-5,6-dihydro-4H-thieno[2,3-c]pyrrol-4-one;
2-(5-fluoro-2-((1-(methylsulfonyl)piperidin-4-yl)amino)pyrimidin-4-yl)-3-methyl-5,6-dihydro-4H-thieno[2,3-c]pyrrol-4-one;
2-(5-Fluoro-2-(((3R,4S)-3-methyl-1-(methylsulfonyl)piperidin-4-yl)amino)pyrimidin-4-yl)-3-methyl-5,6-dihydro-4H-thieno[2,3-c]pyrrol-4-one;
6,6-Dimethyl-2-(2-(((3R,4S)-3-methyl-1-(methylsulfonyl)piperidin-4-yl)amino)-5-(trifluoro-methyl)pyrimidin-4-yl)-5,6-dihydro-4H-thieno[2,3-c]pyrrol-4-one;
6,6-dimethyl-2-(2-(((3R,4S)-3-methyl-1-((1-methyl-1H-pyrazol-4-yl)sulfonyl)piperidin-4-yl)amino)-5-(trifluoromethyl)pyrimidin-4-yl)-5,6-dihydro-4H-thieno[2,3-c]pyrrol-4-one;
6,6-dimethyl-2-(2-(((3R,4S)-3-methyl-1-((1-methyl-1H-imidazol-4-yl)sulfonyl)piperidin-4-yl)amino)-5-(trifluoromethyl)pyrimidin-4-yl)-5,6-dihydro-4H-thieno[2,3-c]pyrrol-4-one;
2-(2-(((3R,4S)-1-((1H-pyrazol-4-yl)sulfonyl)-3-methylpiperidin-4-yl)amino)-5-(trifluoro-omethyl)pyrimidin-4-yl)-6,6-dimethyl-5,6-dihydro-4H-thieno[2,3-c]pyrrol-4-one;
2-(2-(((3R,4S)-1-((1H-imidazol-4-yl)sulfonyl)-3-methylpiperidin-4-yl)amino)-5-(trifluoro-methyl)pyrimidin-4-yl)-6,6-dimethyl-5,6-dihydro-4H-thieno[2,3-c]pyrrol-4-one;
6,6-dimethyl-2-(2-((1-((1-methyl-1H-pyrazol-4-yl)sulfonyl)piperidin-4-yl)amino)-5-(trifluoromethyl)pyrimidin-4-yl)-5,6-dihydro-4H-thieno[2,3-c]pyrrol-4-one;
6,6-dimethyl-2-(2-((1-(methylsulfonyl)piperidin-4-yl)amino)-5-(trifluoromethyl) pyrimidin-4-yl)-5,6-dihydro-4H-thieno[2,3-c]pyrrol-4-one;
2-(2-(((3R,4R)-3-fluoro-1-(methylsulfonyl)piperidin-4-yl)amino)-5-(trifluoromethyl) pyrimidin-4-yl)-6,6-dimethyl-5,6-dihydro-4H-thieno[2,3-c]pyrrol-4-one;
2-(2-(((3R,4S)-3-fluoro-1-(methylsulfonyl)piperidin-4-yl)amino)-5-(trifluoromethyl) pyrimidin-4-yl)-6,6-dimethyl-5,6-dihydro-4H-thieno[2,3-c]pyrrol-4-one;
2-(2-(((3R,4S)-3-fluoro-1-((1-methyl-1H-imidazol-4-yl)sulfonyl)piperidin-4-yl)amino)-5-(trifluoromethyl)pyrimidin-4-yl)-6,6-dimethyl-5,6-dihydro-4H-thieno[2,3-c]pyrrol-4-one;
2-(2-(((3R,4R)-3-fluoro-1-((1-methyl-1H-pyrazol-4-yl)sulfonyl)piperidin-4-yl)amino)-5-(trifluoromethyl)pyrimidin-4-yl)-6,6-dimethyl-5,6-dihydro-4H-thieno[2,3-c]pyrrol-4-one;
2-(2-((1-(methylsulfonyl)piperidin-4-yl)amino)-5-(trifluoromethyl)pyrimidin-4-yl)-5,6-dihydro-4H-thieno[2,3-c]pyrrol-4-one;
2-(2-(((3R,4S)-3-methyl-1-(methylsulfonyl)piperidin-4-yl)amino)-5-(trifluoromethyl) pyrimidin-4-yl)-5,6-dihydro-4H-thieno[2,3-c]pyrrol-4-one;
2-(2-(((3R,4S)-3-fluoro-1-(methylsulfonyl)piperidin-4-yl)amino)-5-(trifluoromethyl) pyrimidin-4-yl)-5,6-dihydro-4H-thieno[2,3-c]pyrrol-4-one;
2-(2-((1-((1-methyl-1H-pyrazol-4-yl)sulfonyl)piperidin-4-yl)amino)-5-(trifluoromethyl) pyrimidin-4-yl)-5,6-dihydro-4H-thieno[2,3-c]pyrrol-4-one;
2-(2-(((3R,4S)-3-methyl-1-((1-methyl-1H-pyrazol-4-yl)sulfonyl)piperidin-4-yl)amino)-5-(trifluoromethyl)pyrimidin-4-yl)-5,6-dihydro-4H-thieno[2,3-c]pyrrol-4-one;
2-(2-(((3R,4S)-1-((1H-pyrazol-4-yl)sulfonyl)-3-fluoropiperidin-4-yl)amino)-5-(trifluoro-methyl)pyrimidin-4-yl)-5,6-dihydro-4H-thieno[2,3-c]pyrrol-4-one;
2-(2-((1-((1-Methyl-1H-pyrazol-4-yl)sulfonyl)piperidin-4-yl)amino)-5-(trifluoromethyl) pyrimidin-4-yl)-4,5-dihydro-6H-thieno[2,3-c]pyrrol-6-one;
2-(2-((1-(Methylsulfonyl)piperidin-4-yl)amino)-5-(trifluoromethyl)pyrimidin-4-yl)-4,5-dihydro-6H-thieno[2,3-c]pyrrol-6-one;
or a pharmaceutically acceptable salt thereof.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein). The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

### Pharmaceutical Compositions and Methods of Administration

In some embodiments, the disclosure is directed to pharmaceutical compositions comprising compounds of Formula I, or a pharmaceutically acceptable salt or solvate thereof.

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-IOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% or more by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)-aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC50). EC50 refers to the plasma concentration required for obtaining 50%> of a maximum effect in vivo.

In some embodiments, the subject methods utilize a CDK inhibitor with an IC50 value of about or less than a predetermined value, as ascertained in an in vitro assay. In some embodiments, the CDK inhibitor inhibits CDK a with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above).

In some embodiments, the CDK inhibitor selectively inhibits CDK a with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above)than its IC50 value against one, two, or three other CDKs.

In some embodiments, the CDK inhibitor selectively inhibits CDK a with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other CDKs.

The subject methods are useful for treating a disease condition associated with CDK. Any disease condition that results directly or indirectly from an abnormal activity or expression level of CDK can be an intended disease condition.

Different disease conditions associated with CDK have been reported. CDK has been implicated, for example, auto-immune diseases, neurodegeneration (such as Parkinson's disease, Alzheimer's disease and ischaemia), inflammatory diseases, viral infections and cancer such as, for example, colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, Head and neck cancer, Heart cancer, Hemoglobinopathies such as b-thalassemia and sickle cell disease (SCD), Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In other embodiments, said method is for treating a disease selected from breast cancer, lung cancer, pancreatic cancer, prostate cancer, colon cancer, ovarian cancer, uterine cancer, or cervical cancer.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS) or epidermoid cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

In some embodiments, the disclosure is directed to methods for treating a CDK4-mediated and a CDK6-mediated disorder in a patient in need thereof, comprising administering to said patient a compound of Formula I, including all subgenera described herein, or a pharmaceutically acceptable salt thereof, or a pharmaceutical composition comprising the compound of Formula I, including all subgenera described herein.

In some embodiments, the CDK4-mediated and CDK6-mediated disorder is a cancer. In some embodiments, the cancer is breast cancer, malignant brain tumors, colon cancer, small-cell lung cancer, non-small-cell lung cancer, bladder cancer, ovarian cancer, prostate cancer, chronic lymphoid leukemia, lymphoma, myeloma, acute myeloid leukemia, secondary pancreatic cancer or secondary brain metastases.

In some embodiments, the cancer is breast cancer. In some embodiments, the cancer is malignant brain tumors. In some embodiments, the cancer is colon cancer. In some embodiments, the cancer is small-cell lung cancer. In some embodiments, the cancer is non-small-cell lung cancer. In some embodiments, the cancer is bladder cancer. In some embodiments, the cancer is ovarian cancer.

In some embodiments, the cancer is prostate cancer. In some embodiments, the cancer is chronic lymphoid leukemia. In some embodiments, the cancer is lymphoma. In some embodiments, the cancer is myeloma. In some embodiments, the cancer is acute myeloid leukemia. In some embodiments, the cancer is secondary pancreatic cancer. In some embodiments, the cancer is secondary brain metastases.

In some embodiments, the breast cancer is HR+/HER2− or HR+/HER2+ advanced or metastatic breast cancer. In some embodiments, the breast cancer is HR+/HER2− advanced breast cancer. In some embodiments, the breast cancer is HR+/HER2− metastatic breast cancer. In some embodiments, the breast cancer is HR+/HER2+ advanced breast cancer. In some embodiments, the breast cancer is HR+/HER2+ metastatic breast cancer.

In some embodiments, the malignant brain tumors are glioblastoma, astrocytoma, or pontine glioma. In some embodiments, the malignant brain tumors are a glioblastoma. In some embodiments, the malignant brain tumors are an astrocytoma. In some embodiments, the malignant brain tumors are a pontine glioma.

In some embodiments, the patient is administered a pharmaceutical composition comprising a compound of Formula I, including all subgenera described herein, or a pharmaceutically acceptable salt thereof. In some embodiments, the administration is oral administration.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds of the invention can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds of the invention can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-Li monoclonal antibody. In some embodiments, the anti-PD-Li monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

For treating autoimmune or inflammatory conditions, the compound of the invention can be administered in combination with a corticosteroid such as triamcinolone, dexamethasone, fluocinolone, cortisone, prednisolone, or flumetholone.

For treating autoimmune or inflammatory conditions, the compound of the invention can be administered in combination with an immune suppressant such as fluocinolone acetonide (Retisert®), rimexolone (AL-2178, Vexol, Alcon), or cyclosporine (Restasis®).

In some embodiments, the disclosure is directed to methods described herein, further comprising administering an additional therapeutic agent to the patient. In some embodiments, the additional therapeutic agent is a PRMT5 inhibitor, a HER2 kinase inhibitor, an aromatase inhibitor, an estrogen receptor antagonist or an alkylating agent.

In some embodiments, the additional therapeutic agent is a PRMT5 inhibitor. In some embodiments, the additional therapeutic agent is a HER2 kinase inhibitor. In other embodiments, the additional therapeutic agent is an aromatase inhibitor. In other embodiments, the additional therapeutic agent is an estrogen receptor antagonist. In yet other embodiments, the additional therapeutic agent is an alkylating agent.

In some embodiments, the aromatase inhibitor is letrozole. In some embodiments, the estrogen receptor antagonist is fulvestrant. In other embodiments, the alkylating agent is temozolomide.

In yet other embodiments, the PRMT5 inhibitor is a compound disclosed in US Published Patent Application No. 2020/0148692 (filed Jan. 16, 2020); US Published Patent Application No. 2019/0284193 (filed Apr. 5, 2019); and US Published Patent Application No. 2019/0048014 (filed Aug. 9, 2018); each of which is hereby incorporated herein in its entirety.

In some embodiments, the PRMT5 inhibitor is:
(2R,3R,4S,5S)-2-(4-amino-7H-pyrrolo[2,3-d]pyrimidin-7-yl)-5-((R)-6-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-7-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-5-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6,7-difluoroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-5,6-difluoroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6-chloro-5-fluoroisochroman-1-yl)tetrahydrofuran-3,4-diol, or a pharmaceutically acceptable salt or solvate thereof;
(2R,3R,4S,5S)-2-(4-amino-5-fluoro-7H-pyrrolo[2,3-d]pyrimidin-7-yl)-5-((R)-6-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6,7-dichloroisochroman-1-yl)tetrahydrofuran-3,4-diol, or a pharmaceutically acceptable salt or solvate thereof;
(2S,3S,4R,5R)-2-((R)-6-chloroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6,7-difluoroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-5,6-difluoroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6-chloroisochroman-1-yl)-5-(5-fluoro-4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6,7-dichloroisochroman-1-yl)-5-(4-methyl-7H-pyrrolo[2,3-d]pyrimidin-7-yl)tetrahydrofuran-3,4-diol, or a pharmaceutically acceptable salt or solvate thereof.
In some embodiments, the PRMT5 inhibitor is (2S,3S,4R,5R)-2-((R)-6-chloroisochroman-1-yl)-5-(4-methyl-7H-pyrrolo[2,3-d]pyrimidin-7-yl)tetrahydrofuran-3,4-diol, or a pharmaceutically acceptable salt or solvate thereof.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.” as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### General Synthetic Procedures

Compounds of Formula (I) can be prepared from optionally protected compounds 1-1 where W¹ is halogen (e.g., Cl, Br, or I) or pseudohalogen (e.g., OTf or OMs) and PG₁ is standard amine protecting groups such as para-methoxybenzyl, as shown in Scheme 1. Compounds 1-1 can be coupled with compounds 1-2 where M¹ is a boronic acid, boronate ester, potassium trifluoroborate, or an appropriately substituted metal, such as Sn(Bu)₃, Sn(Me)₃, or ZnCl, under standard Suzuki conditions (e.g., in the presence of a palladium catalyst, such as [1,1′-bis(diphenyl-phosphino)ferrocene]dichloropalladium(II) and a base, such as K₃PO₄), or standard Stille conditions (e.g., in the presence of a palladium(0) catalyst, such as tetrakis(triphenylphosphine)palladium(0)), or standard Negishi conditions (e.g., in the presence of a palladium catalyst, such as tetrakis (triphenylphosphine)palladium(0) or [1,1′-bis(diphenylphosphino)ferrocene]dichloropalladium(II)), to give compounds 1-3 where W² is halogen (e.g., Cl, Br, or I) or pseudohalogen (e.g., OTf or OMs). Coupling of compounds 1-3 with amines 1-8, with appropriate protecting group (PG₂) (e.g., Boc, etc.), under standard Buchwald-Hartwig amination conditions (e.g., in the presence of a palladium catalyst, such as XPhos Pd G3, and a base, such as Cs₂CO₃ or K₃PO₄) or nucleophilic aromatic substitution conditions (e.g., in the presence of a base such as triethylamine) can provide compounds 1-9. Compounds 1-9 can be deprotected (e.g., TFA, etc.) and subsequently reacted with compounds 1-10 where W⁴ is halogen (e.g., F, Cl or Br) under standard conditions (e.g., in the presence of a base, such as triethylamine) to afford compounds of Formula (I).

Alternatively, compounds 1-1 can be converted to the appropriate compounds 1-4 (e.g., M² is B(OH)₂, Bpin, BF₃K, Sn(Me)₃, Sn(Bu)₃, or ZnCl) and then coupled to compounds 1-5 where W³ is halogen (e.g., Cl, Br, or I) or pseudohalogen (e.g., OTf or OMs) under standard Suzuki conditions (e.g., in the presence of a palladium catalyst, such as [1,1′-bis(diphenylphosphino) ferrocene] dichloropalladium(II), and a base, such as K₃PO₄) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenyl-phosphine)palladium(0) or [1,1′-bis(diphenylphosphino)ferrocene]dichloropalladium(II)) to give compounds 1-3, which can be used to synthesize compounds of Formula (I).

Further alternatively, coupling of compounds 1-6 where W⁵ is halogen (e.g., Cl, Br, or I) or hydrogen with pyrimidines 1-7 in the presence of a strong base (such as LDA, BuLi etc.) and subsequent addition of DDQ can provide the appropriate intermediates 1-3 which can be converted to compounds of Formula (I).

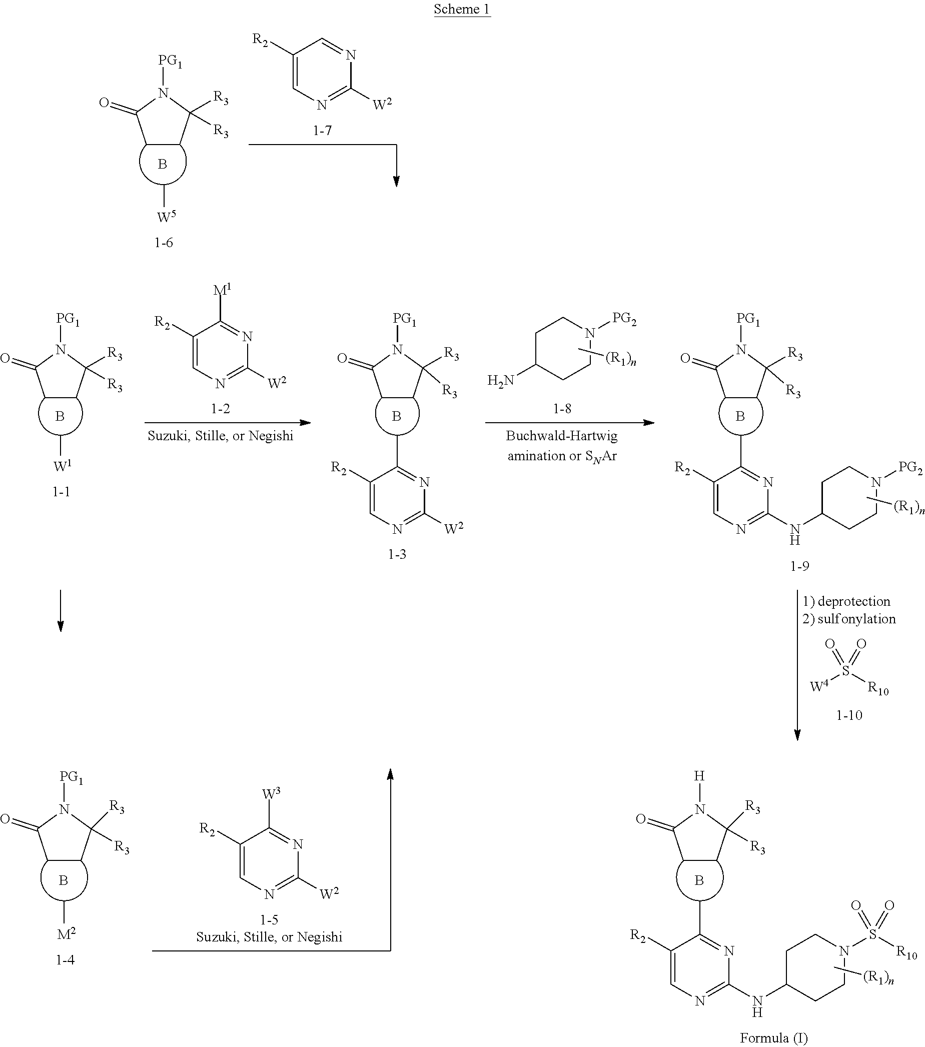

Compounds of Formula (I) can be prepared by the methods described in Scheme 2. Compounds 2-1 where W² is halogen (e.g., Cl, Br, or I) or pseudohalogen (e.g., OTf or OMs) can react with amines 2-2 under amination conditions (e.g., in the presence of a Zn catalyst, such as ZnCl₂, and a base, such as Et₃N) to provide compounds 2-3. Compounds 2-3 where W³ is halogen (e.g., Cl, Br, or I) or pseudohalogen (e.g., OTf or OMs) can then be transformed to compounds of Formula (I) by reacting with compounds 2-4 (e.g., M² is B(OH)₂, Bpin, BF₃K, Sn(Me)₃, Sn(Bu)₃, or ZnCl) under standard Suzuki conditions (e.g., in the presence of a palladium catalyst, such as [1,1′-bis(diphenylphosphino)ferrocene]dichloro-palladium(II), and a base, such as K₃PO₄), or standard Stille conditions (e.g., in the presence of a palladium(0) catalyst, such as tetrakis(triphenyl-phosphine)palladium(0)), or standard Negishi conditions (e.g., in the presence of a palladium(0) catalyst, such as tetrakis(triphenylphosphine) palladium(0) or [1,1′-bis(diphenylphosphino) ferrocene]dichloropalladium(II)). Alternatively, compounds 2-3 can be converted to the appropriate compounds 2-5 (e.g., M³ is B(OH)₂, Bpin, BF₃K, Sn(Me)₃, Sn(Bu)₃, or ZnCl) and then coupled to compounds 2-6 where W¹ is halogen (e.g., Cl, Br, or I) or pseudohalogen (e.g., OTf or OMs) under standard Suzuki conditions (e.g., in the presence of a palladium catalyst, such as [1,1′-bis(diphenyl-phosphino)ferrocene] dichloropalladium(II), and a base, such as K₃PO₄) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenyl-phosphine)palladium(0) or [1,1′-bis(diphenylphosphino)ferrocene]dichloropalladium(II)) to synthesize compounds of Formula (I).

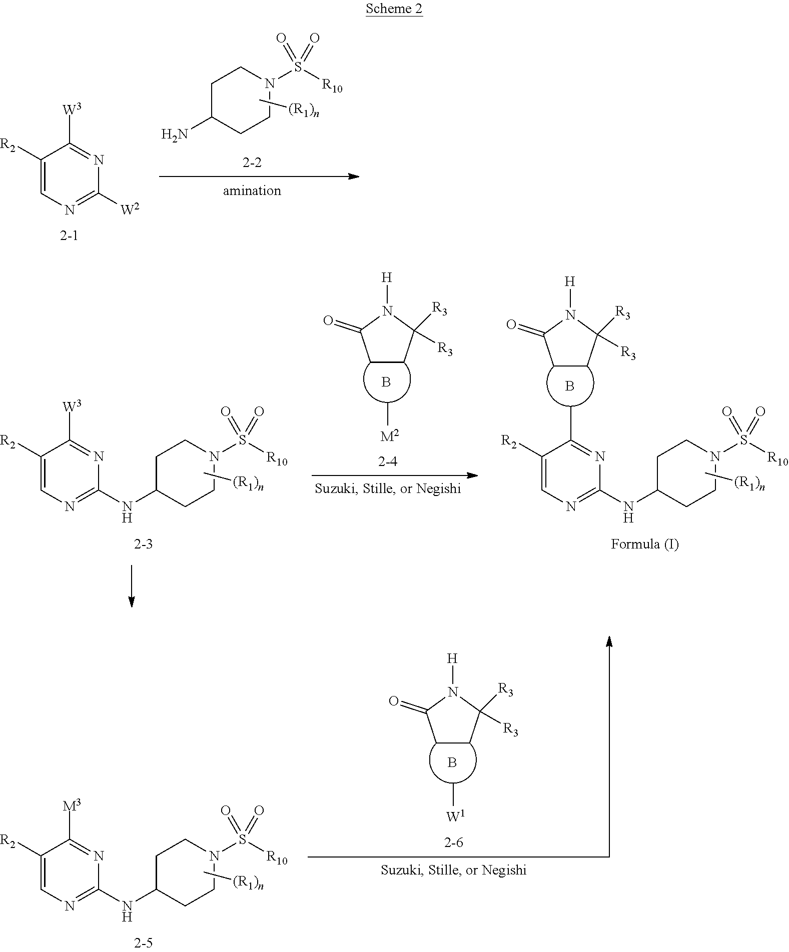

Intermediates for the synthesis of compounds of Formula (I) can be prepared as described in Scheme 3. Compounds 3-1 where W² is halogen (e.g., Cl, Br, or I) or pseudohalogen (e.g., OTf or OMs) can react with amines 3-2 under amination conditions (e.g., in the presence of a Zn catalyst, such as ZnCl₂, and a base, such as Et₃N) to provide compounds 3-3. Compounds 3-3 where W³ is halogen (e.g., Cl, Br, or I) or pseudohalogen (e.g., OTf or OMs) can then be transformed to compounds 3-7 by reacting with compounds 3-4 (e.g., M² is B(OH)₂, Bpin, BF₃K, Sn(Me)₃, Sn(Bu)₃, or ZnCl) under standard Suzuki conditions (e.g., in the presence of a palladium catalyst, such as [1,1′-bis(diphenylphosphino)ferrocene]dichloro-palladium(II), and a base, such as K₃PO₄), or standard Stille conditions (e.g., in the presence of a palladium(0) catalyst, such as tetrakis(triphenylphosphine)palladium(0)), or standard Negishi conditions (e.g., in the presence of a palladium(0) catalyst, such as tetrakis(triphenylphosphine) palladium(0) or [1,1′-bis(diphenyl-phosphino)ferrocene]dichloropalladium(II)). Alternatively, compounds 3-3 can be converted to the appropriate compounds 3-5 (e.g., M³ is B(OH)₂, Bpin, BF₃K, Sn(Me)₃, Sn(Bu)₃, or ZnCl) and then coupled to compounds 3-6 where W¹ is halogen (e.g., Cl, Br, or I) or pseudohalogen (e.g., OTf or OMs) under standard Suzuki conditions (e.g., in the presence of a palladium catalyst, such as [1,1′-bis(diphenylphosphino)ferrocene] dichloropalladium(II), and a base, such as K₃PO₄) or standard Stille conditions (e.g., in the presence of a palladium(0) catalyst, such as tetrakis(triphenyl-phosphine)palladium(0)) or standard Negishi conditions (e.g., in the presence of a palladium catalyst, such as tetrakis(triphenylphosphine)palladium(0) or [1,1′-bis(diphenylphosphino) ferrocene]dichloropalladium(II)) to synthesize compounds 3-7.

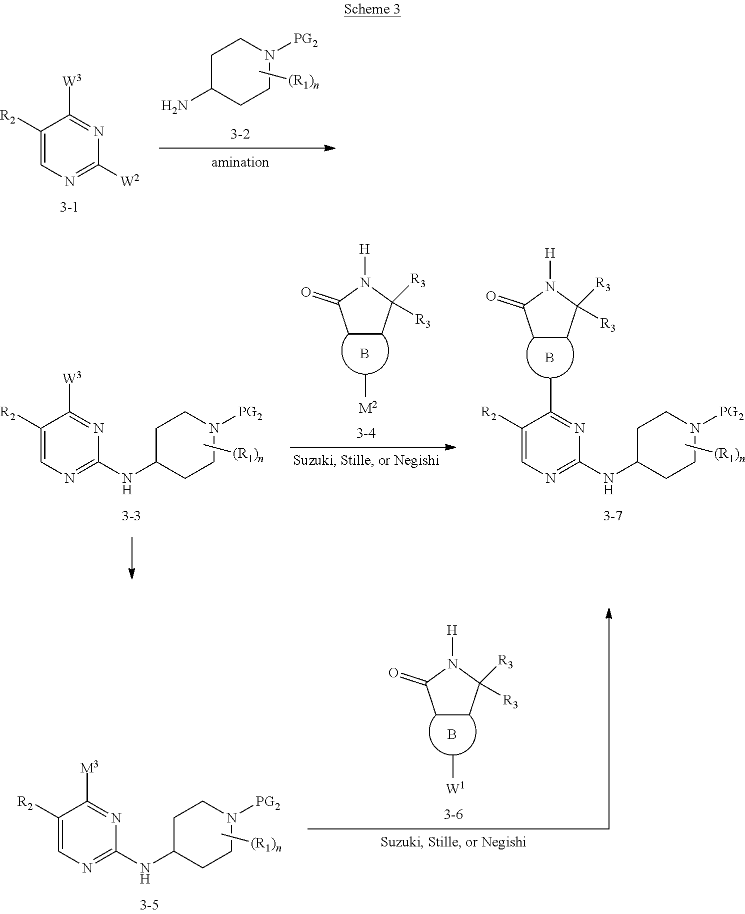

Intermediates for the synthesis of compounds of Formula (I) can be prepared as described in Scheme 4. Optionally protected compounds 4-1 where PG₁ is standard amine protecting groups such as para-methoxybenzyl can be halogenated with suitable reagents, such as N-bromosuccinimide or N-iodosuccinimide, to provide compounds 4-2. Alternatively, compounds 4-1 can be metalated in the presence of a strong base, such as lithium diisopropylamide or butyllithium, and an appropriate reagent (e.g., 2-isopropoxy-4,4,5,5-tetramethyl-1,3,2-dioxaborolane, hexamethylditin, trimethyltin chloride, or zinc chloride) to afford compounds 4-3.

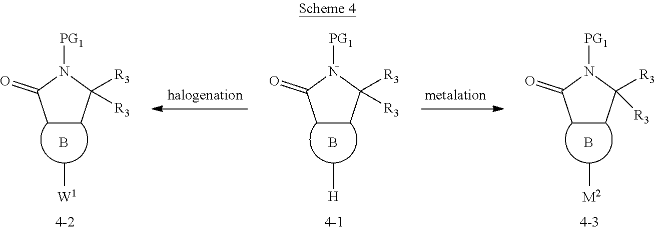

Intermediates for the synthesis of compounds of Formula (I) can be prepared as described in Scheme 5. Lithiation of acids 5-1 (W⁴ is H, Br, or I) and reaction with N-sulfonyl imines 5-2 can provide the corresponding acids 5-3 which can be converted to the lactams 5-4 under the amide coupling conditions in the presence of a coupling reagent (e.g., N,N′-dicyclohexyl-carbodiimide or HATU, etc.). Removal of the sulfinamide group can yield lactams 5-5.

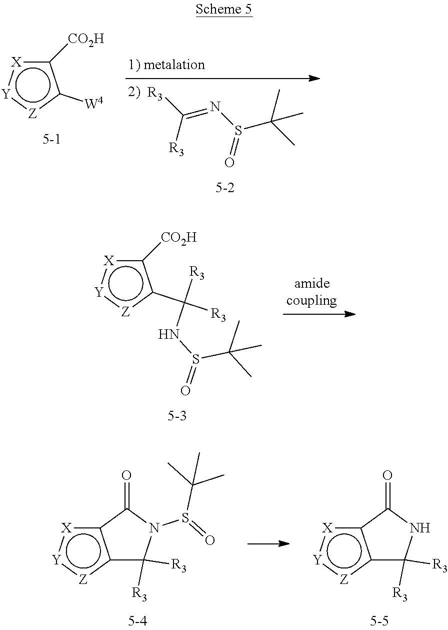

Alternatively, intermediates 6-5 can be prepared as described in Scheme 6. Lithiation of acids 6-1 (W⁴ is H, Br, or I) with a strong base, such as butyllithium, and subsequent addition into ketones 6-2 can afford alcohols 6-3. Lactone formation under standard conditions, such as in the presence of benzenesulfonyl chloride and pyridine or in the presence of an acid (e.g., tosic acid) or in the presence of a coupling reagent (e.g., N,N′-dicyclohexylmethanediimine or HATU) and optionally an additive (e.g., 1-hydroxybenzotriazole) and optionally a base (e.g., pyridine or N,N-diisopropylethylamine) can afford lactones 6-4. Exposure of compounds 6-4 to ammonia under standard conditions, such as at elevated temperature and pressure, can provide lactams 6-5.

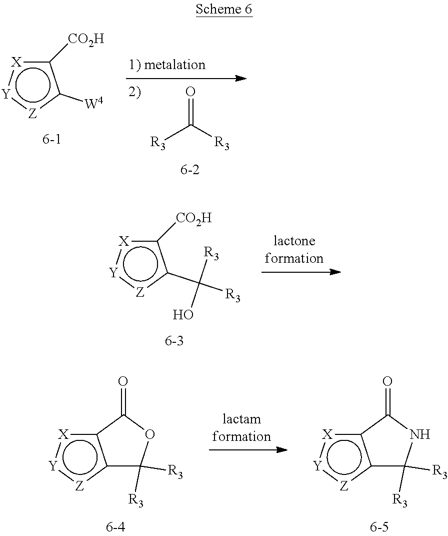

Alternatively, intermediates 7-8 can be prepared as the methods described in Scheme 7. Amide coupling of acids 7-1 (W⁴ is H, Br, or I) with a suitable amines NH₂—PG₁ where PG₁ is standard amine protecting groups such as para-methoxybenzyl in the presence of a coupling reagent (e.g., N,N′-dicyclohexylcarbodiimide or HATU) and optionally an additive (e.g., 1-hydroxybenzotriazole or 4-dimethylaminopyridine) and optionally a base (e.g., pyridine or N,N-diisopropylethylamine) can provide amides 7-2. Lithiation of compounds 7-2 with a strong base, such as butyllithium, and subsequent addition into a formyl transfer reagent such as N,N-dimethyl-formamide, N-methoxy-N-methylformamide or N-formylmorpholine can afford aldehydes 7-3. Reduction of aldehydes 7-3 under standard conditions, such as in the presence of a reducing agent (e.g., sodium borohydride, lithium aluminumhydride, or borane tetrahydrofuran complex), can provide alcohols 7-4 which can be halogenated with suitable reagents, such as thionyl chloride or phosphorus tribromide or triphenylphosphine and iodine, to provide compounds 7-5 where X^{c} is a halogen (e.g., Cl, Br, or I). Ring closure reaction of compounds 7-5 under basic conditions (e.g., in the presence of sodium hydride or lithium bis(trimethylsilyl)amide) can afford lactams 7-6. Bis-alkylation of lactams 7-6 in the presence of a base, such as sodium hydride, can afford compounds 7-7. Deprotection of compounds 7-7 under appropriate conditions can provide intermediates 7-8.

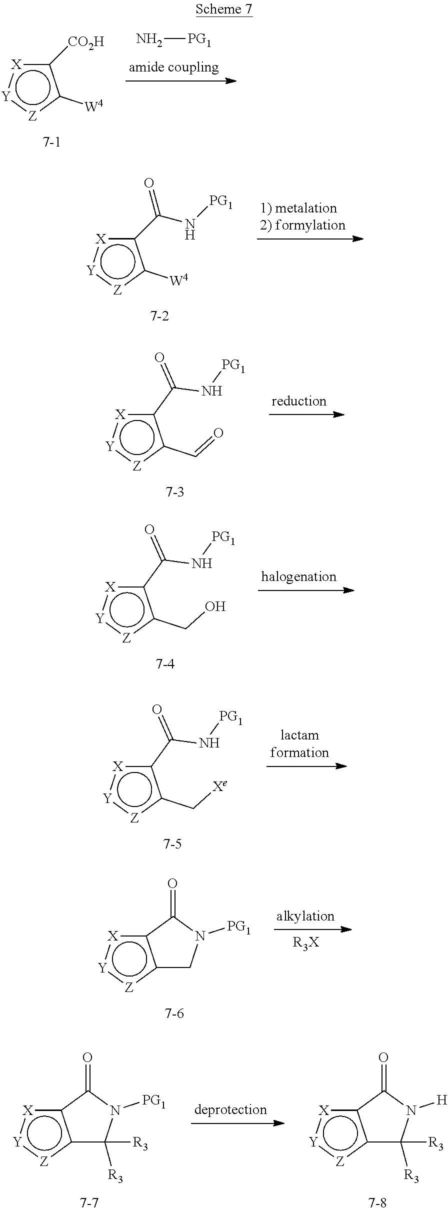

### Example 1. 2-(5-Fluoro-2-(((3R,4S)-3-methyl-1-(methylsulfonyl)piperidin-4-yl)amino) pyrimidin-4-yl)-3,6,6-trimethyl-5,6-dihydro-4H-thieno[2,3-c]pyrrol-4-one

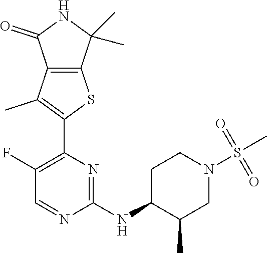 [SMILES: Cc1c(-c2nc(N[C@H]3CCN(S(C)(=O)=O)C[C@H]3C)ncc2F)sc2c1C(=O)NC2(C)C]

### Step 1. N-[(4-Methoxyphenyl)methyl]-4-methylthiophene-3-carboxamide

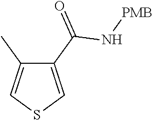

To a solution of 4-methyl-3-thiophenecarboxylic acid (15.0 g, 105 mmol) in THF (200 mL) was added 1-ethyl-3-(3′-dimethylaminopropyl)carbodiimide hydrochloride (13.9 g, 137 mmol). The mixture was stirred at room temperature for 30 min, and then 4-methoxybenzylamine (15.9 g, 116 mmol) was added dropwise at 0° C. The reaction was slowly warmed to room temperature and stirred at room temperature for 2 h. The reaction mixture was poured into 1 N HCl (aq.) (100 mL) and extracted by DCM (100 mL×3). The combined organic layers were washed with brine (200 mL), dried over Na₂SO₄, filtered, and concentrated under reduced pressure. The crude product was purified by silica gel chromatography (2-35% EtOAc/heptane) to afford the title compound (15.5 g, 59.3 mmol, 56.2% yield). LCMS calc. for C₁₄H₁₆NO₂S [M+H]⁺: m/z=262.09; Found: 262.1.

### Step 2. 2-Formyl-N-[(4-methoxyphenyl)methyl]-4-methylthiophene-3-carboxamide

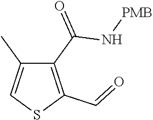

A solution of n-butyllithium (48.5 mL, 121 mmol, 2.5 M in hexanes) was added dropwise to a solution of N-[(4-methoxyphenyl)methyl]-4-methylthiophene-3-carboxamide (14.4 g, 55.1 mmol) in THF (250 mL) at −78° C. under N₂ atmosphere. The reaction was stirred at −78° C. for 25 min followed by addition of DMF (25.5 mL, 331 mmol) dropwise. The reaction was slowly warmed to room temperature and stirred at room temperature for 1 h. The reaction was quenched with cold sat. NH₄Cl (aq.) (100 mL) and extracted with EtOAc (100 mL×3). The combined organic layers were dried over Na₂SO₄, filtered, and concentrated to yield the title compound, which was used without further purification. LCMS calc. for C₁₅H₁₆NO₃S [M+H]⁺: m/z=290.09; Found: 290.0.

### Step 3. 2-(Hydroxymethyl)-N-[(4-methoxyphenyl)methyl]-4-methylthiophene-3-carboxamide

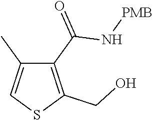

To a crude solution of 2-formyl-N-[(4-methoxyphenyl)methyl]-4-methylthiophene-3-carboxamide (from Step 2) in methanol (250 mL) was added sodium borohydride (2.08 g, 55.0 mmol) portion-wise at 0° C. The reaction was warmed to rt and stirred for 90 min. The reaction was quenched with water (200 mL) and filtered. The precipitate was collected and washed with 1:1 MeOH/H₂O (50 mL), re-dissolved in DCM (100 mL) and dried over Na₂SO₄, filtered, and concentrated to yield the title compound (13.4 g, 46.0 mmol, 83.6% yield over 2 steps). LCMS calc. for C₁₅H₁₇NNaO₃S[M+Na]⁺: m/z=314.08; Found: 314.0. LCMS calc. for C₁₅H₁₆NO₂S [M−OH]⁺: 274.09; Found: 274.0.

### Step 4. 2-(Chloromethyl)-N-[(4-methoxyphenyl)methyl]-4-methylthiophene-3-carboxamide

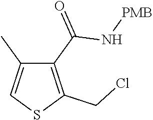

To a solution of 2-(hydroxymethyl)-N-[(4-methoxyphenyl)methyl]-4-methylthiophene-3-carboxamide (10.0 g, 34.3 mmol) in DCM (200 mL) was added thionyl chloride (2.74 mL, 37.7 mmol) dropwise at 0° C. The reaction was slowly warmed to rt and stirred for 15 min. The reaction mixture was poured into cold sat. NaHCO₃ (aq.) (100 mL) and extracted with DCM (100 mL×3). The combined organic layers were dried over Na₂SO₄, filtered, and concentrated to afford the title compound, which was used without further purification. LCMS calc. for C₁₅H₁₆NO₂S [M-Cl]⁺: m/z=274.09; Found: 274.0.

### Step 5. 5-[(4-Methoxyphenyl)methyl]-3-methyl-6H-thieno[2,3-c]pyrrol-4-one

To a crude solution of 2-(chloromethyl)-N-[(4-methoxyphenyl)methyl]-4-methylthiophene-3-carboxamide (from Step 4) in THF (1800 mL) was added a solution of lithium bis(trimethylsilyl)amide (37.2 mL, 46.5 mmol, 1.25 M in THF) dropwise at −78° C. The reaction was slowly warmed to rt over 1 h. The reaction was poured into cold sat. NH₄Cl (aq.) (100 mL). The mixture was concentrated and extracted with EtOAc (100 mL×3). The combined organic layers were dried over Na₂SO₄, filtered, and concentrated. The residue was purified by silica gel chromatography (10-70% EtOAc/heptane) to afford the title compound (8.50 g, 28.3 mmol, 73.1% yield over 2 steps). LCMS calc. for C₁₅H₁₆NO₂S [M+H]⁺: m/z=274.1; Found 274.1.

### Step 6. 5-(4-Methoxybenzyl)-3,6,6-trimethyl-5,6-dihydro-4H-thieno[2,3-c]pyrrol-4-one

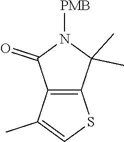

To a solution of 5-[(4-methoxyphenyl)methyl]-3-methyl-6H-thieno[2,3-c]pyrrol-4-one (1.10 g, 4.02 mmol) in DMF (3 mL) was added sodium hydride (483 mg, 12.1 mmol). The reaction mixture was stirred at rt for 30 min. Next iodomethane (0.750 mL, 12.2 mmol) was added. The resulting mixture was further stirred at rt overnight. LCMS showed the starting material was consumed. The reaction was quenched with saturated NH₄Cl solution (10 mL) and extracted with EtOAc (10 mL×3). The combined organic layers were washed with brine, dried over Na₂SO₄, filtered, and concentrated. The residue was purified by silica gel chromatography (0-20% EtOAc/heptane) to afford the title compound (953 mg, 3.15 mmol, 78.6% yield). LCMS calc. for C₁₇H₂₀NO₂S [M+H]⁺: m/z=302.12; Found 302.1.

### Step 7. 2-Bromo-5-(4-methoxybenzyl)-3,6,6-trimethyl-5,6-dihydro-4H-thieno[2,3-c]pyrrol-4-one

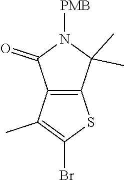

To a solution of 5-[(4-methoxyphenyl)methyl]-3,6,6-trimethylthieno[2,3-c]pyrrol-4-one (950 mg, 3.15 mmol) in MeCN (10 mL) was added N-bromosuccinimide (610 mg, 3.47 mmol). The reaction mixture was stirred at rt for 30 min. LCMS showed the starting material was consumed. The solvent was removed under reduced pressure. The residue was purified by silica gel chromatography (0-20% EtOAc/heptane) to afford the title compound (1.00 g, 2.63 mmol, 83.4% yield). LCMS calc. for C₁₇H₁₉BrNO₂S [M+H]⁺: m/z=382.03, 380.03; Found 381.9, 380.0.

### Step 8. 2-(2-Chloro-5-fluoropyrimidin-4-yl)-5-(4-methoxybenzyl)-3,6,6-trimethyl-5,6-dihydro-4H-thieno[2,3-c]pyrrol-4-one

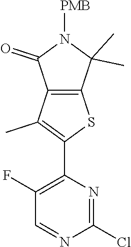

To a solution of 2-bromo-5-[(4-methoxyphenyl)methyl]-3,6,6-trimethylthieno[2,3-c]pyrrol-4-one (1.00 g, 2.63 mmol) and hexamethylditin (1.09 mL, 5.26 mmol) in 1,4-dioxane (150 mL) was added tetrakis(triphenylphosphine)palladium(0) (304 mg, 0.263 mmol). The reaction mixture was sparging with N₂ for 10 min and stirred at 100° C. under N₂ atmosphere overnight. LCMS showed the starting material was consumed. The reaction was cooled to rt, then 2,6-dichloro-5-fluororacil (967 mg, 5.78 mmol) and tetrakis(triphenylphosphine)palladium(0) (304 mg, 0.263 mmol) were added. The reaction mixture was sparged with N₂ for 10 min and stirred at 100° C. under N₂ atmosphere for 48 h. LCMS showed the intermediate was consumed. The reaction was quenched with KF solution and stirred at rt for 1 h. The reaction mixture was filtered, and the filtrate was concentrated under reduced pressure. The residue was purified by silica gel chromatography (0-20% EtOAc/heptane) once, and further purified by prep-HPLC on a C18 column (20-70% MeCN in 0.1% TFA(aq), pH=2) to afford the title compound (515 mg, 1.19 mmol, 45.3% yield). LCMS calc. for C₂₁H₂₀ClFN₃O₂S [M+H]⁺: m/z=432.09; Found 432.0.

### Step 9. 2-(2-Chloro-5-fluoropyrimidin-4-yl)-3,6,6-trimethyl-5,6-dihydro-4H-thieno[2,3-c]pyrrol-4-one

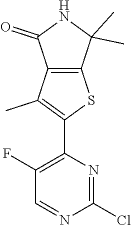 [SMILES: Cc1c(-c2nc(Cl)ncc2F)sc2c1C(=O)NC2(C)C]

A solution of 2-(2-chloro-5-fluoropyrimidin-4-yl)-5-(4-methoxybenzyl)-3,6,6-trimethyl-5,6-dihydro-4H-thieno[2,3-c]pyrrol-4-one (530 mg, 1.23 mmol) in trifluoroacetic acid (5.0 mL) was stirred at 75° C. for 3 h. The reaction mixture was cooled to rt and concentrated under reduced pressure. The residue was purified by prep-HPLC on a C18 column (20-50% MeCN in 0.1% TFA (aq.), pH=2) to afford the title compound (230 mg, 0.738 mmol, 60.1%). LCMS calc. for C₁₃H₁₂ClFN₃OS [M+H]⁺: m/z=312.04; Found 312.0.

### Step 10. Tert-butyl (3R,4S)-4-((5-fluoro-4-(3,6,6-trimethyl-4-oxo-5,6-dihydro-4H-thieno[2,3-c]pyrrol-2-yl)pyrimidin-2-yl)amino)-3-methylpiperidine-1-carboxylate

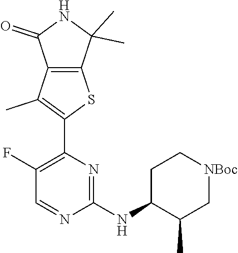 [SMILES: Cc1c(-c2nc(N[C@H]3CCN(C(=O)OC(C)(C)C)C[C@H]3C)ncc2F)sc2c1C(=O)NC2(C)C]

A solution of 2-(2-chloro-5-fluoropyrimidin-4-yl)-3,6,6-trimethyl-5,6-dihydro-4H-thieno[2,3-c]pyrrol-4-one (60.0 mg, 0.192 mmol), tert-butyl (3R,4S)-4-amino-3-methylpiperidine-1-carboxylate (49.5 mg, 0.231 mmol) and N,N-diisopropylethylamine (0.335 mL, 1.92 mmol) in NMP (1.0 mL) was stirred at 145° C. for 48 h. The reaction mixture was cooled to rt, diluted with MeCN and purified by prep-HPLC on a C18 column (20-50% MeCN in 0.1% TFA (aq.), pH=2) to afford the title compound (45.0 mg, 0.0919 mmol, 47.8%). LCMS calc. for C₂₄H₃₃FN₅O₃S [M+H]⁺: m/z=490.23; Found 490.5.

### Step 11. 2-(5-Fluoro-2-(((3R,4S)-3-methylpiperidin-4-yl)amino)pyrimidin-4-yl)-3,6,6-trimethyl-5,6-dihydro-4H-thieno[2,3-c]pyrrol-4-one

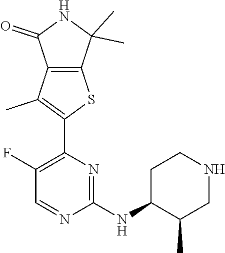 [SMILES: Cc1c(-c2nc(N[C@H]3CCNC[C@H]3C)ncc2F)sc2c1C(=O)NC2(C)C]

To a solution of tert-butyl (3R,4S)-4-[[5-fluoro-4-(3,6,6-trimethyl-4-oxo-5H-thieno[2,3-c]pyrrol-2-yl)pyrimidin-2-yl]amino]-3-methylpiperidine-1-carboxylate (45.0 mg, 0.0919 mmol) in DCM (0.5 mL) was added trifluoroacetic acid (0.2 mL). The reaction mixture was stirred at rt for 15 min. LCMS showed the starting material was consumed. The solvent was removed under reduced pressure to afford the title compound (36.0 mg, assume quantitative yield) as a crude, which was used directly for the next reaction without further purification. LCMS calc. for C₁₉H₂₅FN₅OS [M+H]⁺: m/z=390.18; Found 390.2.

### Step 12. 2-(5-Fluoro-2-(((3R,42)-3-methyl-1-(methylsulfonyl)piperidin-4-yl)amino) pyrimidin-4-yl)-3,6,6-trimethyl-5,6-dihydro-4H-thieno[2,3-c]pyrrol-4-one

To a solution of 2-[5-fluoro-2-[[(3R,4S)-3-methylpiperidin-4-yl]amino]pyrimidin-4-yl]-3,6,6-trimethyl-5H-thieno[2,3-c]pyrrol-4-one (36.0 mg, 0.0924 mmol) and N,N-diisopropylethylamine (0.048 mL, 0.28 mmol) in DMA (1.0 mL) was added methanesulfonyl chloride (0.0086 mL, 0.11 mmol) at 0° C. The reaction mixture was stirred at rt for 15 min LCMS showed the starting material was consumed. The reaction was diluted with MeOH (1 mL) and purified by prep-HPLC on a C18 column (20-50% MeCN in 0.1% TFA (aq.), pH=2) to afford the title compound (29.3 mg, 0.0504 mmol, 54.5% yield) as its TFA salt. LCMS calc. for C₂₀H₂₇FN₅O₃S₂ [M+H]⁺: m/z=468.15; Found 468.0. ¹H NMR (300 MHz, DMSO-d₆) δ 8.51 (s, 1H), 8.49-8.45 (m, 1H), 7.44-7.39 (m, 1H), 4.11-4.02 (m, 1H), 3.34-2.99 (m, 4H), 2.86 (s, 3H), 2.53-2.45 (m, 3H), 2.25-2.11 (m, 1H), 1.87-1.60 (m, 2H), 1.51 (s, 6H), 0.95-0.84 (in, 3H).

### Example 2-5

Example 2-5 listed in Table 1 were prepared in accordance with the synthetic protocols set forth in Example 1 using appropriate intermediates and reagents.

TABLE 1
Example 2-5

| Example | | | [M + 1] |
|---|---|---|---|
| # | Structure | Name | Calc./found |
| 2 | 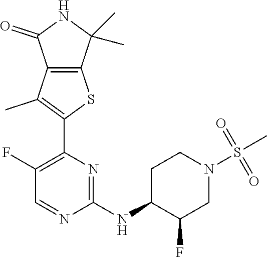 [SMILES: Cc1c(-c2nc(N[C@H]3CCN(S(C)(=O)=O)C[C@H]3F)ncc2F)sc2c1C(=O)NC2(C)C] | 2-(5-fluoro-2-(((3R,4S)-3-fluoro-1- (methylsulfonyl) piperidin-4- yl)amino)pyrimidin-4-yl)-3,6,6- trimethyl-5,6-dihydro-4H- thieno[2,3-c]pyrrol-4-one | 472.13/472.0 |
| 3 | 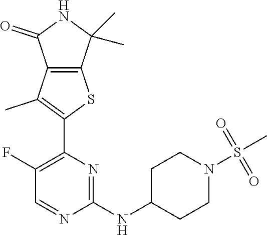 [SMILES: Cc1c(-c2nc(NC3CCN(S(C)(=O)=O)CC3)ncc2F)sc2c1C(=O)NC2(C)C] | 2-(5-fluoro-2-((1-(methylsulfonyl) piperidin-4-yl)amino)pyrimidin-4- yl)-3,6,6-trimethyl-5,6-dihydro-4H- thieno[2,3-c]pyrrol-4-one | 454.14/454.1 |
| 4 | 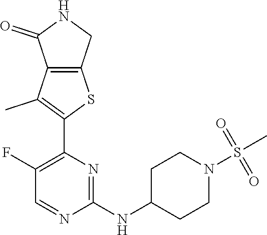 [SMILES: Cc1c(-c2nc(NC3CCN(S(C)(=O)=O)CC3)ncc2F)sc2c1C(=O)NC2] | 2-(5-fluoro-2-((1-(methylsulfonyl) piperidin-4-yl)amino) pyrimidin-4- yl)-3-methyl-5,6-dihydro-4H-thieno [2,3-c]pyrrol-4-one | 426.11/425.9 |
| 5 | 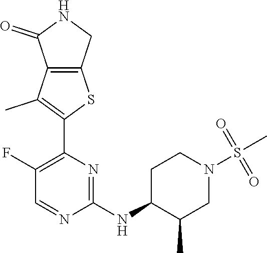 [SMILES: Cc1c(-c2nc(N[C@H]3CCN(S(C)(=O)=O)C[C@H]3C)ncc2F)sc2c1C(=O)NC2] | 2-(5-Fluoro-2-(((3R,4S)-3-methyl-1- (methylsulfonyl)piperidin-4-yl) amino)pyrimidin-4-yl)-3-methyl- 5,6-dihydro-4H-thieno[2,3-c]pyrrol- 4-one | 440.12/440.0 |

### Intermediate 1. 2-Bromo-6,6-dimethyl-5,6-dihydro-4H-thieno[2,3-c]pyrrol-4-one

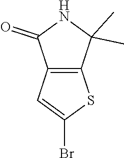

The title compound was purchased from commercial sources (eNovation Chemicals, catalog #: Y1003616). Synthesis of the title compound was reported (WO2019/223632).

### Intermediate 2. 2-Bromo-5,6-dihydro-4H-thieno[2,3-c]pyrrol-4-one

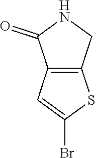

N-Bromosuccinimide (569 mg, 3.23 mmol) was added to a solution of 5,6-dihydro-4H-thieno[2,3-c]pyrrol-4-one (300 mg, 2.16 mmol) in acetonitrile (5.0 mL) and stirred at rt overnight. The mixture was concentrated. The crude residue was purified by silica gel column chromatography (0-30% EtOAc in heptanes) to afford the title compound (215 mg, 0.985 mmol, 45.6% yield). LCMS calc. for C₆H₅BrNOS [M+H]⁺: m/z=217.93, 219.93; Found 217.9, 219.8.

### Intermediate 3. Tert-butyl (3R,4S)-4-((4-chloro-5-(trifluoromethyl)pyrimidin-2-yl)amino)-3-methylpiperidine-1-carboxylate

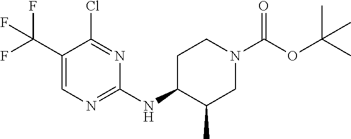 [SMILES: C[C@@H]1CN(C(=O)OC(C)(C)C)CC[C@@H]1Nc1ncc(C(F)(F)F)c(Cl)n1]

To a solution of 2,4-dichloro-5-(trifluoromethyl)pyrimidine (1.10 g, 4.73 mmol) in DCE (12 mL)/tert-butanol (12 mL) was added zinc chloride (890 mg, 6.52 mmol) at 0° C. The mixture was stirred for 1 h, followed by the dropwise addition of tert-butyl (3R,4S)-4-amino-3-methyl-piperidine-1-carboxylate (1.00 g, 4.73 mmol) and triethylamine (538 mg, 5.31 mmol) at 0° C. The mixture was then stirred at 40° C. overnight. LCMS showed that the starting material was consumed. The solvent was removed under reduced pressure. The mixture was then added H₂O (10 mL) and extracted with ethyl acetate (10 mL×3). The combined organic layers were washed with brine, dried over Na₂SO₄, filtered, and concentrated. The crude was purified by prep-HPLC on C18 column (15-70% MeCN in 0.1% TFA(aq), pH=2) to afford the title compound (1.10 g, 2.78 mmol, 59.7% yield). LCMS calc. for C₁₆H₂₃ClF₃N₆O₂ [M+H]⁺: m/z=395.15; Found 395.1.

### Intermediates 4-6

Intermediates 4-6 listed in Table 2 were prepared in accordance with the synthetic protocols set forth in Intermediate 3 using the appropriate amines.

TABLE 2
Intermediates 4-6

| Intermediate | | |
|---|---|---|
| # | Structure and name | Analytical Data |
| 4 | 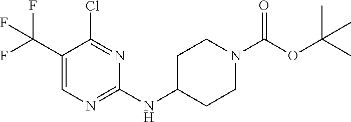 [SMILES: CC(C)(C)OC(=O)N1CCC(Nc2ncc(C(F)(F)F)c(Cl)n2)CC1] Tert-butyl 4-((4-chloro-5-(trifluoro- methyl)pyrimidin-2-yl)amino) piperidine-1-carboxylate | LCMS calc. for C₁₅H₂₁ClF₃N₄O₂ [M + H]⁺: m/z = 381.13; Found 381.1. |
| 5 | 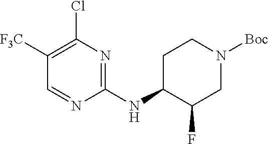 [SMILES: CC(C)(C)OC(=O)N1CC[C@H](Nc2ncc(C(F)(F)F)c(Cl)n2)[C@H](F)C1] Tert-Butyl (3R,4S)-4-((4-chloro-5- (trifluoromethyl)pyrimidin-2-yl) amino)-3-fluoropiperidine-1- carboxylate | ¹H NMR (300 MHz, CDCl₃) δ 8.45 (s, 1H), 5.90-5.80 (m, 1H), 4.85- 4.70 (m, 1H), 4.50-4.40 (m, 1H), 4.30-4.20 (m, 1H), 3.15-2.80 (m, 2H), 1.90-1.80 (m, 2H), 1.48 (s, 9H). |
| 6 | 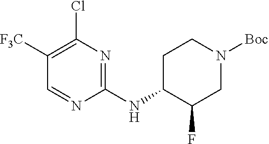 [SMILES: CC(C)(C)OC(=O)N1CC[C@@H](Nc2ncc(C(F)(F)F)c(Cl)n2)[C@H](F)C1] Tert-butyl (3R,4R)-4-((4-chloro-5- (trifluoromethyl)pyrimidin-2-yl) amino)-3-fluoropiperidine-1- carboxylate | ¹H NMR (300 MHz, CDCl₃) δ 8.46 (s, 1H), 5.65-5.60 (m, 1H), 4.50- 4.25 (m, 3H), 4.00-3.90 (m, 1H), 3.10-3.00 (m, 2H), 2.25-2.00 (m, 1H), 1.48 (s, 9H). |

### Example 6. 6,6-Dimethyl-2-(2-(((3R,4S)-3-methyl-1-(methylsulfonyl)piperidin-4-yl)amino)-5-(trifluoromethyl)pyrimidin-4-yl)-5,6-dihydro-4H-thieno[2,3-c]pyrrol-4-one

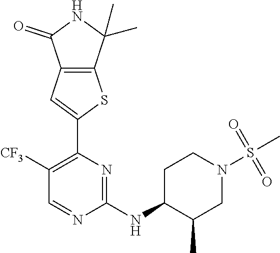 [SMILES: C[C@@H]1CN(S(C)(=O)=O)CC[C@@H]1Nc1ncc(C(F)(F)F)c(-c2cc3c(s2)C(C)(C)NC3=O)n1]

### Step 1. Tert-butyl (3R,4S)-4-((4-(6,6-dimethyl-4-oxo-5,6-dihydro-4H-thieno[2,3-c]pyrrol-2-yl)-5-(trifluoromethyl)pyrimidin-2-yl)amino)-3-methylpiperidine-1-carboxylate

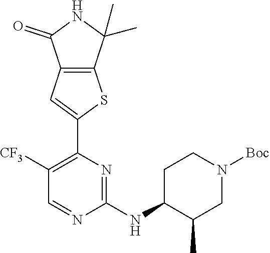 [SMILES: C[C@@H]1CN(C(=O)OC(C)(C)C)CC[C@@H]1Nc1ncc(C(F)(F)F)c(-c2cc3c(s2)C(C)(C)NC3=O)n1]

A mixture of 2-bromo-6,6-dimethyl-5,6-dihydro-4H-thieno[2,3-c]pyrrol-4-one (Intermediate 1) (40.0 mg, 0.163 mmol), Pd(PPh₃)₄(37.6 mg, 0.0325 mmol), tert-butyl (3R,4S)-4-((4-chloro-5-(trifluoromethyl)pyrimidin-2-yl)amino)-3-methylpiperidine-1-carboxylate (Intermediate 3) (64.2 mg, 0.163 mmol), hexamethylditin (0.0674 mL, 0.325 mmol) in 1,4-dioxane (18 mL) was sparging with N₂ for 5 min. The mixture was then stirred at 100° C. in a sealed tube for 12 h. LCMS showed the starting material was consumed. The reaction was then cooled to rt and potassium fluoride (5 mL, 10% aq.) was added. The mixture was stirred at rt for 1 h. The solvent was then removed under reduced pressure. The residue was extracted with ethyl acetate (10 mL×2). The combined organic layers were washed with brine, dried over Na₂SO₄, filtered, and concentrated. The crude was purified by prep-HPLC on C18 column (10-70% MeCN in 0.1% TFA(aq), pH=2) to afford the title compound (81.2 mg, 94.8% yield). LCMS calc. for C₂₄H₃₁F₃N₅O₃S [M+H]⁺: m/z=526.21; Found 526.1.

### Step 2. 6,6-Dimethyl-2-(2-(((3R,4S)-3-methylpiperidin-4-yl)amino)-5-(trifluoro-methyl)pyrimidin-4-yl)-5,6-dihydro-4H-thieno[2,3-c]pyrrol-4-one

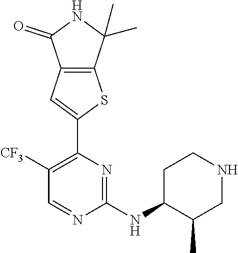 [SMILES: C[C@@H]1CNCC[C@@H]1Nc1ncc(C(F)(F)F)c(-c2cc3c(s2)C(C)(C)NC3=O)n1]

To a solution of tert-butyl (3R,4S)-4-((4-(6,6-dimethyl-4-oxo-5,6-dihydro-4H-thieno[2,3-c]pyrrol-2-yl)-5-(trifluoromethyl)pyrimidin-2-yl)amino)-3-methylpiperidine-1-carboxylate (81.2 mg, 0.154 mmol) in DCM (4 mL) was added TFA (2 mL). The mixture was stirred at rt for 1 h. LCMS showed the starting material was consumed. The reaction mixture was concentrated under reduced pressure to afford the title compound (65.7 mg, quantitative yield) which was directly used in the next step without further purification. LCMS calc. for C₁₉H₂₃F₃N₅OS [M+H]⁺: m/z=426.16; Found 426.0.

### Step 3. 6,6-Dimethyl-2-(2-(((3R,4S)-3-methyl-1-(methylsulfonyl)piperidin-4-yl)amino)-5-(trifluoromethyl)pyrimidin-4-yl)-5,6-dihydro-4H-thieno[2,3-c]pyrrol-4-one

To a solution of 6,6-dimethyl-2-(2-(((3R,4S)-3-methylpiperidin-4-yl)amino)-5-(trifluoro-methyl)pyrimidin-4-yl)-5,6-dihydro-4H-thieno[2,3-c]pyrrol-4-one (60.0 mg, 0.141 mmol) in DMA (1.0 mL) was added methanesulfonyl chloride (0.012 mL, 0.155 mmol) at 0° C., followed by the addition of N,N-diisopropylethylamine (0.245 mL, 1.41 mmol). The mixture was then stirred at rt for 3 h. LCMS showed the starting material was consumed. The crude was purified by prep-HPLC on C18 column (10-60% MeCN in 0.1% TFA(aq), pH=2) to afford the title compound (43.5 mg, 50.0% yield) as its TFA salt. LCMS calc. for C₂₀H₂₅F₃N₅O₃S₂ [M+H]⁺: m/z=504.14; Found 504.0. ¹H NMR (300 MHz, DMSO-d₆) δ 8.79-8.58 (m, 2H), 8.39-8.08 (m, 1H), 7.54 (d, J=18.3 Hz, 1H), 4.29-4.10 (m, 1H), 3.40-2.99 (m, 4H), 2.95-2.79 (m, 3H), 2.27-2.09 (m, 1H), 1.93-1.66 (m, 2H), 1.60-1.47 (m, 6H), 0.95-0.82 (in, 3H).

### Examples 7-22

Examples 7-22 listed in Table 3 were prepared in accordance with the synthetic protocols set forth in Example 1 using the appropriate intermediates and reagents.

TABLE 3
Examples 7-22

| Example | | | [M + 1] |
|---|---|---|---|
| # | Structure | name | Calc./found |
| 7 | 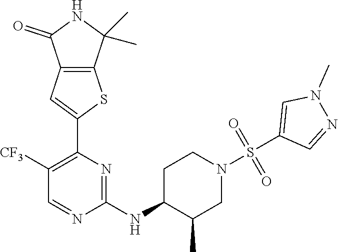 [SMILES: C[C@@H]1CN(S(=O)(=O)c2cnn(C)c2)CC[C@@H]1Nc1ncc(C(F)(F)F)c(-c2cc3c(s2)C(C)(C)NC3=O)n1] | 6,6-dimethyl-2-(2-(((3R,4S)- 3-methyl-1-((1-methyl-1H- pyrazol-4-yl)sulfonyl) piperidin-4-yl)amino)-5- (trifluoromethyl)pyrimidin- 4-yl)-5,6-dihydro-4H- thieno[2,3-c]pyrrol-4-one | 570.16/570.1 |
| 8 | 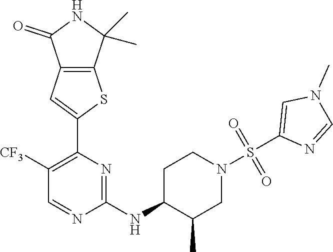 [SMILES: C[C@@H]1CN(S(=O)(=O)c2cn(C)cn2)CC[C@@H]1Nc1ncc(C(F)(F)F)c(-c2cc3c(s2)C(C)(C)NC3=O)n1] | 6,6-dimethyl-2-(2-(((3R,4S)- 3-methyl-1-((1-methyl-1H- imidazol-4-yl)sulfonyl) piperidin-4-yl)amino)-5- (trifluoromethyl)pyrimidin- 4-yl)-5,6-dihydro-4H- thieno[2,3-c]pyrrol-4-one | 570.16/570.4 |
| 9 | 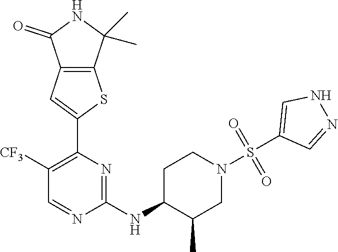 [SMILES: C[C@@H]1CN(S(=O)(=O)c2cn[nH]c2)CC[C@@H]1Nc1ncc(C(F)(F)F)c(-c2cc3c(s2)C(C)(C)NC3=O)n1] | 2-(2-(((3R,4S)-1-((1H- pyrazol-4-yl)sulfonyl)-3- methylpiperidin-4-yl) amino)-5-(trifluoro- methyl)pyrimidin-4-yl)-6,6- dimethyl-5,6-dihydro-4H- thieno[2,3-c]pyrrol-4-one | 556.14/556.1 |
| 10 | 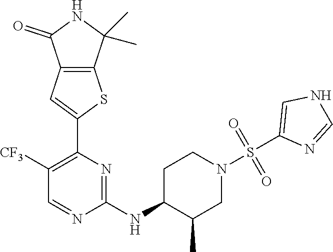 [SMILES: C[C@@H]1CN(S(=O)(=O)c2c[nH]cn2)CC[C@@H]1Nc1ncc(C(F)(F)F)c(-c2cc3c(s2)C(C)(C)NC3=O)n1] | 2-(2-(((3R,4S)-1-((1H- imidazol-4-yl)sulfonyl)-3- methylpiperidin-4-yl) amino)-5-(trifluoro- methyl)pyrimidin-4-yl)-6,6- dimethyl-5,6-dihydro-4H- thieno[2,3-c]pyrrol-4-one | 556.14/556.2 |
| 11 | 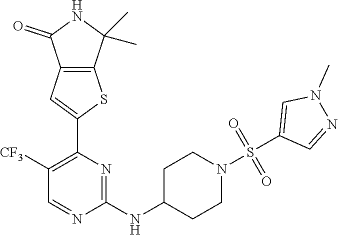 [SMILES: Cn1cc(S(=O)(=O)N2CCC(Nc3ncc(C(F)(F)F)c(-c4cc5c(s4)C(C)(C)NC5=O)n3)CC2)cn1] | 6,6-dimethyl-2-(2-((1-((1- methyl-1H-pyrazol-4-yl) sulfonyl)piperidin-4-yl) amino)-5-(trifluoro-methyl) pyrimidin-4-yl)-5,6-dihydro- 4H-thieno[2,3-c]pyrrol-4- one | 556.14/556.1 |
| 12 | 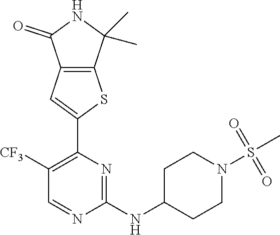 [SMILES: CC1(C)NC(=O)c2cc(-c3nc(NC4CCN(S(C)(=O)=O)CC4)ncc3C(F)(F)F)sc21] | 6,6-dimethyl-2-(2-((1- (methylsulfonyl)piperidin-4- yl)amino)-5-(trifluoro- methyl)pyrimidin-4-yl)-5,6- dihydro-4H-thieno[2,3-c] pyrrol-4-one | 490.12/490.2 |
| 13 | 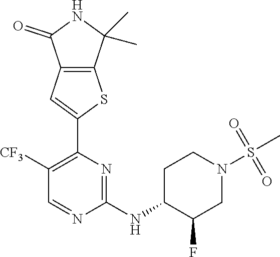 [SMILES: CC1(C)NC(=O)c2cc(-c3nc(N[C@@H]4CCN(S(C)(=O)=O)C[C@H]4F)ncc3C(F)(F)F)sc21] | 2-(2-(((3R,4R)-3-fluoro-1- (methylsulfonyl)piperidin-4- yl)amino)-5-(trifluoro- methyl)pyrimidin-4-yl)-6,6- dimethyl-5,6-dihydro-4H- thieno[2,3-c]pyrrol-4-one | 508.11/508.1 |
| 14 | 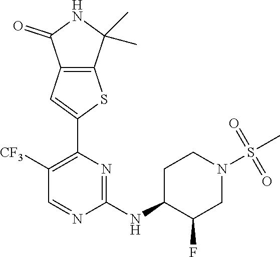 [SMILES: CC1(C)NC(=O)c2cc(-c3nc(N[C@H]4CCN(S(C)(=O)=O)C[C@H]4F)ncc3C(F)(F)F)sc21] | 2-(2-(((3R,4S)-3-fluoro-1- (methylsulfonyl)piperidin-4- yl)amino)-5-(trifluoro- methyl)pyrimidin-4-yl)-6,6- dimethyl-5,6-dihydro-4H- thieno[2,3-c]pyrrol-4-one | 508.11/508.1 |
| 15 | 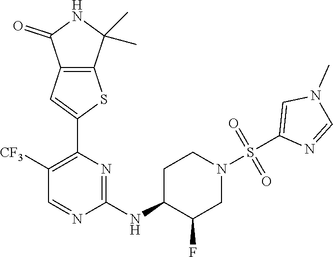 [SMILES: Cn1cnc(S(=O)(=O)N2CC[C@H](Nc3ncc(C(F)(F)F)c(-c4cc5c(s4)C(C)(C)NC5=O)n3)[C@H](F)C2)c1] | 2-(2-(((3R,4S)-3-fluoro-1- ((1-methyl-1H-imidazol-4- yl)sulfonyl)piperidin-4- yl)amino)-5-(trifluoro- methyl)pyrimidin-4-yl)-6,6- dimethyl-5,6-dihydro-4H- thieno[2,3-c]pyrrol-4-one | 574.13/574.2 |
| 16 | 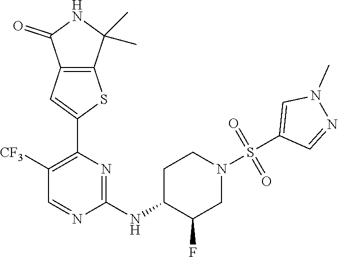 [SMILES: Cn1cc(S(=O)(=O)N2CC[C@@H](Nc3ncc(C(F)(F)F)c(-c4cc5c(s4)C(C)(C)NC5=O)n3)[C@H](F)C2)cn1] | 2-(2-(((3R,4R)-3-fluoro-1- ((1-methyl-1H-pyrazol-4- yl)sulfonyl)piperidin-4- yl)amino)-5-(trifluoro- methyl)pyrimidin-4-yl)-6,6- dimethyl-5,6-dihydro-4H- thieno[2,3-c]pyrrol-4-one | 574.13/574.1 |
| 17 | 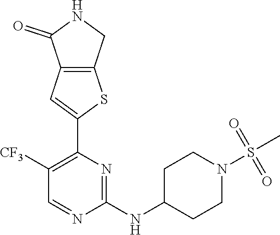 [SMILES: CS(=O)(=O)N1CCC(Nc2ncc(C(F)(F)F)c(-c3cc4c(s3)CNC4=O)n2)CC1] | 2-(2-((1-(methylsulfonyl) piperidin-4-yl)amino)-5- (trifluoromethyl)pyrimidin- 4-yl)-5,6-dihydro-4H- thieno[2,3-c]pyrrol-4-one | 462.09/462.1 |
| 18 | 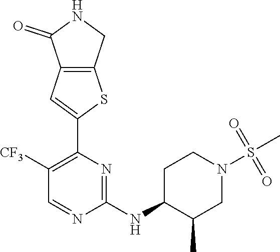 [SMILES: C[C@@H]1CN(S(C)(=O)=O)CC[C@@H]1Nc1ncc(C(F)(F)F)c(-c2cc3c(s2)CNC3=O)n1] | 2-(2-(((3R,4S)-3-methyl-1- (methylsulfonyl)piperidin-4- yl)amino)-5-(trifluoro- methyl)pyrimidin-4-yl)-5,6- dihydro-4H-thieno[2,3- c]pyrrol-4-one | 476.10/476.0 |
| 19 | 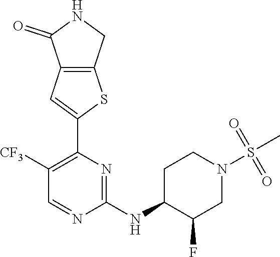 [SMILES: CS(=O)(=O)N1CC[C@H](Nc2ncc(C(F)(F)F)c(-c3cc4c(s3)CNC4=O)n2)[C@H](F)C1] | 2-(2-(((3R,4S)-3-fluoro-1- (methylsulfonyl) piperidin- 4-yl)amino)-5-(trifluoro- methyl)pyrimidin-4-yl)-5,6- dihydro-4H-thieno[2,3- c]pyrrol-4-one | 480.08/479.9 |
| 20 | 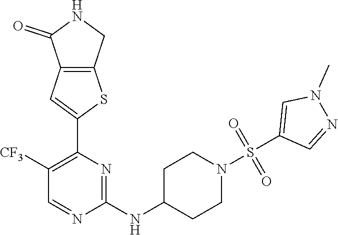 [SMILES: Cn1cc(S(=O)(=O)N2CCC(Nc3ncc(C(F)(F)F)c(-c4cc5c(s4)CNC5=O)n3)CC2)cn1] | 2-(2-((1-((1-methyl-1H- pyrazol-4-yl)sulfonyl) piperidin-4-yl)amino)-5- (trifluoromethyl)pyrimidin- 4-yl)-5,6-dihydro-4H- thieno[2,3-c]pyrrol-4-one | 528.11/528.1 |
| 21 | 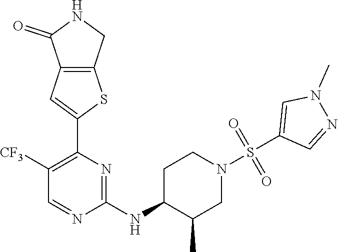 [SMILES: C[C@@H]1CN(S(=O)(=O)c2cnn(C)c2)CC[C@@H]1Nc1ncc(C(F)(F)F)c(-c2cc3c(s2)CNC3=O)n1] | 2-(2-(((3R,4S)-3-methyl-1- ((1-methyl-1H-pyrazol-4- yl)sulfonyl)piperidin-4- yl)amino)-5-(trifluoro- methyl)pyrimidin-4-yl)-5,6- dihydro-4H-thieno[2,3- c]pyrrol-4-one | 542.13/542.0 |
| 22 | 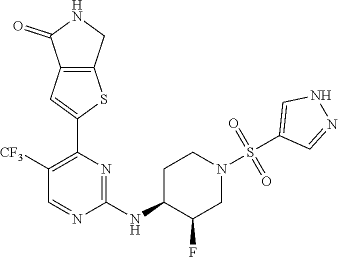 [SMILES: O=C1NCc2sc(-c3nc(N[C@H]4CCN(S(=O)(=O)c5cn[nH]c5)C[C@H]4F)ncc3C(F)(F)F)cc21] | 2-(2-(((3R,4S)-1-((1H- pyrazol-4-yl)sulfonyl)-3- fluoropiperidin-4-yl)amino)- 5-(trifluoromethyl) pyrimidin-4-yl)-5,6-dihydro- 4H-thieno[2,3-c]pyrrol-4- one | 532.08/532.0 |

### Intermediate 7. 2-Bromo-4,5-dihydro-6H-thieno[2,3-c]pyrrol-6-one

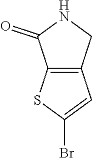

The title compound was purchased from commercial sources (Astatech, catalog #: 94083). Synthesis of the title compound was reported (WO 2016/057500).

### Intermediate 8. 4-Chloro-N-(1-((1-methyl-1H-pyrazol-4-yl)sulfonyl)piperidin-4-yl)-5-(trifluoromethyl)pyrimidin-2-amine

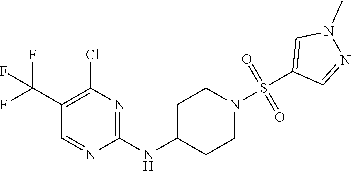 [SMILES: Cn1cc(S(=O)(=O)N2CCC(Nc3ncc(C(F)(F)F)c(Cl)n3)CC2)cn1]

### Step 1. 4-Chloro-N-(piperidin-4-yl)-5-(trifluoromethyl)pyrimidin-2-amine

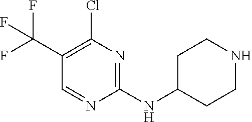 [SMILES: FC(F)(F)c1cnc(NC2CCNCC2)nc1Cl]

The title compound was synthesized by a procedure analogous to that outlined in Example 6, Step 2, using tert-butyl 4-[[4-chloro-5-(trifluoromethyl)pyrimidin-2-yl]amino]piperidine-1-carboxylate (Intermediate 4) as starting material. LCMS calc. for C₁₀H₁₃ClF₃N₄ [M+H]⁺: m/z=281.08; Found 281.2.

### Step 2. 4-Chloro-N-(1-((1-methyl-1H-pyrazol-4-yl)sulfonyl)piperidin-4-yl)-5-(trifluoromethyl)pyrimidin-2-amine

The title compound was synthesized by a procedure analogous to that outlined in Example 6, Step 3, using 4-chloro-N-(piperidin-4-yl)-5-(trifluoromethyl)pyrimidin-2-amine as starting material, and 1-methyl-1H-pyrazole-4-sulfonyl chloride as reagent. LCMS calc. for C₁₄H₁₇ClF₃N₆O₂S [M+H]⁺: m/z=425.1; Found 425.0.

### Example 23. 2-(2-((1-((1-Methyl-1H-pyrazol-4-yl)sulfonyl)piperidin-4-yl)amino)-5-(trifluoromethyl)pyrimidin-4-yl)-4,5-dihydro-6H-thieno[2,3-c]pyrrol-6-one

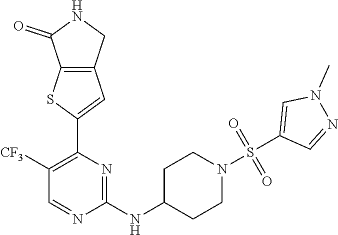 [SMILES: Cn1cc(S(=O)(=O)N2CCC(Nc3ncc(C(F)(F)F)c(-c4cc5c(s4)C(=O)NC5)n3)CC2)cn1]

The title compound was synthesized by a procedure analogous to that outlined in Example 6, Step 1, using 2-bromo-4,5-dihydro-6H-thieno[2,3-c]pyrrol-6-one (Intermediate 7) and 4-chloro-N-(1-((1-methyl-1H-pyrazol-4-yl)sulfonyl)piperidin-4-yl)-5-(trifluoromethyl)pyrimidin-2-amine (Intermediate 8) as starting material. ¹H NMR (300 MHz, DMSO-d₆) δ 8.75-8.56 (m, 2H), 8.34 (d, J=5.1 Hz, 1H), 8.31-8.07 (m, 1H), 7.86-7.60 (m, 2H), 4.36 (s, 2H), 3.92 (d, J=3.8 Hz, 3H), 3.87-3.70 (m, 1H), 3.50 (d, J=11.5 Hz, 2H), 2.61-2.33 (m, 2H), 2.12-1.87 (m, 2H), 1.64 (dd, J=21.9, 9.7 Hz, 2H). LCMS calc. for C₂₀H₂₁F₃N₇O₃S₂ [M+H]⁺: m/z=528.11; Found 528.1.

### Example 24. 2-(2-((1-(Methylsulfonyl)piperidin-4-yl)amino)-5-(trifluoromethyl)pyrimidin-4-yl)-4,5-dihydro-6H-thieno[2,3-c]pyrrol-6-one

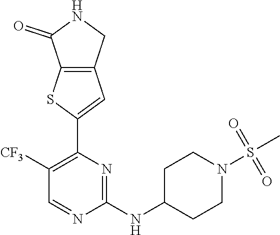 [SMILES: CS(=O)(=O)N1CCC(Nc2ncc(C(F)(F)F)c(-c3cc4c(s3)C(=O)NC4)n2)CC1]

The title compound was synthesized by a procedure analogous to that outlined in Example 23. ¹H NMR (300 MHz, DMSO-d6) δ 8.83-8.57 (m, 2H), 8.42-8.05 (m, 1H), 7.73 (d, J=15.5 Hz, 1H), 4.37 (s, 2H), 4.06-3.85 (m, 1H), 3.68-3.52 (m, 2H), 3.04-2.78 (m, 5H), 2.09-1.94 (m, 2H), 1.72-1.51 (m, 2H). LCMS calc. for C₁₇H₁₉F₃N₅O₃S₂ [M+H]⁺: m/z=462.09; Found 462.0.

### Example A: Enzymatic Activity and Cytotoxicity Studies

### CDK2/CyclinE2 Enzymatic Activity Assay

The inhibitory activity of compounds was evaluated in vitro using TR-FRET assay with white 384-well low volume microplate (Greiner Bio-One). CDK2/Cyclin E2 catalyzed phosphorylation of peptide in the presence and absence of compounds was measured and used in IC₅₀ determination. Recombinant protein complex CDK2/Cyclin E2, expressed from insect cell, was purchased from ProQinase. Testing compounds were dissolved in DMSO at 1 mM and tested in 9-dose IC₅₀ mode. The reaction mixture was prepared by mixing CDK2/CyclinE2 (1 nM final), ULight-4E-BP1 (50 nM final, Perkinelmer, TRF0128-D), and ATP (1 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by dispenser (TECAN D300E) to make a 9.9 μL of reaction mixture. After 20 minutes preincubation at room temperature, 0.1 μL MgCl₂ (10 mM final) was added to initiate the reaction. Following a 45 minutes incubation at 37° C., the reaction was stopped by addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubate at room temperature for additional 60 minutes in dark. The reaction signal was measured by Envision multimode plate reader (PerkinElmer, 2102-0010). IC₅₀ values were determined by fitting the data to the standard 4 parameters with Hill Slope using GraphPad Prism software.

### CDK4/CyclinD1 Enzymatic Activity Assay

The inhibitory activity of compounds was evaluated in vitro using TR-FRET assay with white 384-well low volume microplate (Greiner Bio-One). CDK4/Cyclin D1 catalyzed phosphorylation of peptide in the presence and absence of compounds was measured and used in IC₅₀ determination. Recombinant protein complex CDK4/Cyclin D1, expressed from insect cell, was purchased from ProQinase. Testing compounds were dissolved in DMSO at 1 mM and tested in 9-dose IC₅₀ mode. The reaction mixture was prepared by mixing CDK4/CyclinD1 (1 nM final), ULight-4E-BP1 (100 nM final, Perkinelmer, TRF0128-D), and ATP (2 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by dispenser (TECAN D300E) to make a 9.9 μL of reaction mixture. After 20 minutes preincubation at room temperature, 0.1 μL MgCl₂ (10 mM final) was added to initiate the reaction. Following a 45 minutes incubation at 37° C., the reaction was stopped by addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubate at room temperature for additional 60 minutes in dark. The reaction signal was measured by Envision multimode plate reader (PerkinElmer, 2102-0010). IC₅₀ values were determined by fitting the data to the standard 4 parameters with Hill Slope using GraphPad Prism software.

### CDK6/CyclinD1 Enzymatic Activity Assay

The inhibitory activity of compounds was evaluated in vitro using TR-FRET assay with white 384-well low volume microplate (Greiner Bio-One). CDK6/CyclinD1 catalyzed phosphorylation of peptide in the presence and absence of compounds was measured and used in IC₅₀ determination. Recombinant protein complex CDK6/CyclinD1 expressed from insect cell, was purchased from ProQinase. Testing compounds were dissolved in DMSO at 1 mM and tested in 9-dose IC₅₀ mode. The reaction mixture was prepared by mixing CDK6/CyclinD1 (1 nM final), ULight-4E-BP1 (100 nM final, Perkinelmer, TRF0128-D), and ATP (1 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by dispenser (TECAN D300E) to make a 9.9 μL of reaction mixture. After 20 minutes preincubation at room temperature, 0.1 μL MgCl₂ (10 mM final) was added to initiate the reaction. Following a 40 minutes incubation at 37° C., the reaction was stopped by addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubate at room temperature for additional 60 minutes in dark. The reaction signal was measured by Envision multimode plate reader (PerkinElmer, 2102-0010). IC₅₀ values were determined by fitting the data to the standard 4 parameters with Hill Slope using GraphPad Prism software.

### Cell Proliferation Studies in OVCAR3 Cells

Cell proliferation studies were conducted in OVCAR3 adenocarcinoma cell line. Cells were maintained in RPMI (Corning, Catalog #: 10-040-CV) supplemented with 10% v/v FBS (Gibco, Catalog #: 26140-079), 1% v/v Penicillin Streptomycin (Gibco, Catalog #15140-122) Cells were seeded in 384-well plates at a density of 250 cells/well. Compounds dissolved in DMSO were plated in quadruplicate using a digital dispenser (D300E, Tecan) and tested on a 9-point 3-fold serial dilution. Cells were incubated for 10 days in a 37° C. active humidified incubator at 5% CO₂. A media exchange and second compound addition were performed on day 5. Cell viability was measured using the ATP-Lite 1-Step Luminescence reagent (Perkin Elmer, Catalog #: 6016731) as per manufacturer's instructions. Luminescence signal was measured with a multimode plate reader (Envision 2105, Perkin Elmer). Raw data files were imported to Dotmatics Screening Ultra for IC₅₀ analysis. Luminescence values were normalized to both background and DMSO controls to obtain a percentage of viable cells relative to DMSO vehicle control.

### pRB ICW Assay in OVCAR3 Cells

OVCAR3 cells were maintained in RPMI (Corning, Catalog #: 10-040-CV) supplemented with 10% v/v FBS (Gibco, Catalog #: 26140-079), 1% v/v Penicillin Streptomycin (Gibco, Catalog #15140-122.) OVCAR3 cells grown at log phase were trypsinized, counted, and resuspended in fresh medium to reach a final density of 6.7e4 cells/ml and 75 μL of culture were dispensed into a 384-well plate (Falcon, cat #353962) using Multidrop Combi dispenser (Thermo Scientific). The next day, compounds were dispensed as a 9-point, ½ log serial dilution using a Tecan digital dispenser (D300e), and cells were incubated with compound for 2 hours in a humidified incubator at 37° C. A reference compound at a final concentration of 10 μM was used as a control for maximum inhibition. Each compound was tested in duplicates in each experiment. At the end of the incubation, 25 μl of 16% paraformaldehyde (Electron Microscopy, cat #15710) was slowly added to each well and the plate was incubated at room temperature (RT) for 30 minutes to fix cells. Cells were then permeabilized by incubating with 50 μl/well of wash buffer (1×PBS with 0.1% Triton X-100) 5×5 minutes, followed by 1 hour blocking with 30 μl/well of Odyssey blocking buffer (Li—COR, cat #927-40000), all at RT. Anti-phosphor RB antibody (Cell signaling 8516S) was diluted 1:1000 in Odyssey blocking buffer and 20 μl was added to all wells and incubated overnight in 4° C. with gently rocking. The next day, cells were washed 5×5 min with 50 μl/well of wash buffer, followed with 1 hour incubation with secondary antibody and DRAQ5 diluted in Odyssey blocking buffer (1:500 dilution for secondary antibody and 1:2000 dilution for DRAQ5), 5×5 min washes, and one last wash with water. Plates were dried in 37° C. oven for 5 min and scanned using Li—COR Odyssey CLx imaging system to acquire intensities at 700 and 800 nm channels.

The IC₅₀ values are summarized in Table 4.

TABLE 4
IC₅₀ Values

| | CDK2_E1 | CDK4_D1 | OVCAR3 p-RB ICW |
|---|---|---|---|
| Example | IC₅₀ (nM) | IC₅₀ (nM) | IC₅₀ (nM) |
| 1 | +++ | +++ | +++ |
| 2 | +++ | +++ | +++ |
| 3 | +++ | +++ | +++ |
| 4 | +++ | ND | +++ |
| 5 | +++ | ND | +++ |
| 6 | +++ | +++ | +++ |
| 7 | +++ | +++ | +++ |
| 8 | +++ | +++ | +++ |
| 9 | +++ | +++ | +++ |
| 10 | +++ | +++ | +++ |
| 11 | +++ | +++ | +++ |
| 12 | +++ | +++ | +++ |
| 13 | +++ | +++ | +++ |
| 14 | +++ | +++ | +++ |
| 15 | +++ | +++ | +++ |
| 16 | +++ | +++ | +++ |
| 17 | +++ | +++ | +++ |
| 18 | +++ | ND | ++ |
| 19 | +++ | ND | +++ |
| 20 | +++ | +++ | +++ |
| 21 | +++ | +++ | +++ |
| 22 | +++ | ND | +++ |
| 23 | +++ | ++ | ++ |
| 24 | +++ | + | + |

In Table 5, a “+” denotes an IC₅₀ value of >500 nM; a “++” denotes an IC₅₀ value of 100 nM<IC₅₀≤500 nM; a “+++” denotes an IC₅₀ value of IC₅₀≤100 nM; ND=not determined.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula I: 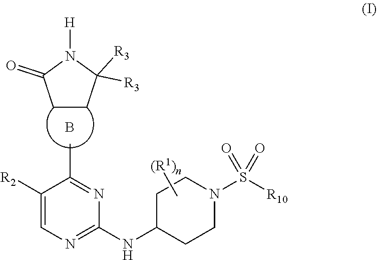
or a pharmaceutically acceptable salt or solvate or N-oxide thereof, wherein ring B is a 5-membered heteroaryl selected from: 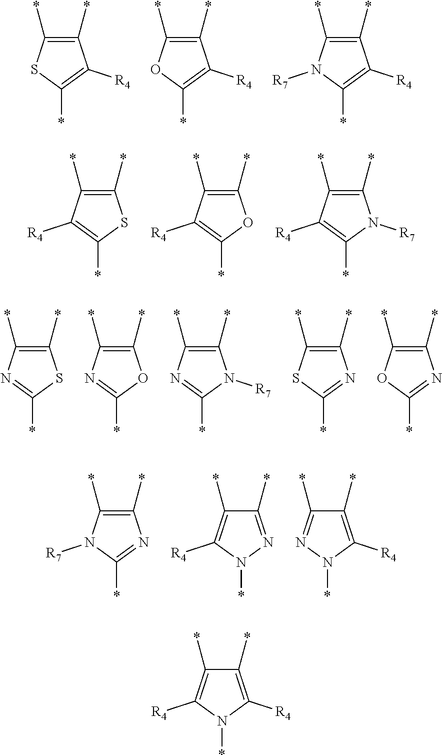
n is 0, 1, 2, 3, 4, 5, 6, 7, 8 or 9;
each R₁, when present, is independently H, D, halogen, —OH, —CN, —NO₂, —C₁-C₆ alkyl, C₁₋₆alkoxide, —C₂-C₆ alkenyl, —C₂-C₆ alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or two R¹ groups together with the atoms to which they are both attached form a carbocyclic or heterocyclic group;
R₂ is H, D, halogen, C₁-C₈ alkoxide, C₁-C₈ alkyl, haloalkoxide, C₁-C₈ haloalkyl, SF₅, or CN, wherein the C₁₋₈ alkyl is optionally substituted with D, halogen, —OH, —CN, or cycloalkyl;
each R₃ is independently H, D, halogen, —OH, —CN, —NO₂, —C₁-C₆ alkyl, —C₂-C₆ alkenyl, —C₂-C₆ alkynyl, C₀-C₁ alk-aryl, C₀-C₁ alk-heteroaryl, cycloalkyl, cycloalkenyl, heterocycloalkyl, heterocycloalkenyl, —OR^{a}, —SR^{b}, —NR^{c}R^{d}, —NR^{a}R^{c}, —C(O)R^{b}, —OC(O)R^{b}, —C(O)OR^{b}, —C(O)NR^{c}R^{d}, or —B(OR^{d})(OR^{c}); wherein the —C₁-C₆ alkyl, —C₂-C₆ alkenyl, —C₂-C₆ alkynyl, C₀-C₁ alk-aryl, C₀-C₁ alk-heteroaryl, cycloalkyl, cycloalkenyl, heterocycloalkyl and heterocycloalkenyl are optionally substituted with at least one of D, halogen, —OH, —CN, an amine, cycloalkyl, or heterocycloalkyl;
each R₄ is independently H, D, halogen, C₁-C₈ alkoxide, C₁-C₈ alkyl, C₃-C₈ cycloalkyl, C₄-C₈ heterocyclyl, —C(O)NR^{c}R^{d}, SF₅ or CN, wherein the C₁₋₈ alkyl, C₃-C₈ cycloalkyl, or C₄-C₈ heterocyclyl are optionally substituted with D, halogen, —OH, —CN, or cycloalkyl;
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, SiR^{b}₃, —C₁-C₁₀ alkyl, —C₂-C₁₀ alkenyl, —C₂-C₁₀ alkynyl, C₀-C₁ alk-aryl, cycloalkyl, cycloalkenyl, C₀-C₁ alk-heteroaryl, heterocycloalkyl, or heterocycloalkenyl;
each R^{b} is independently H, D, —C₁-C₆ alkyl, —C₂-C₆ alkenyl, —C₂-C₆ alkynyl, C₀-C₁ alk-aryl, cycloalkyl, cycloalkenyl, C₀-C₁ alk-heteroaryl, heterocycloalkyl, or heterocycloalkenyl;
each R^{c} is independently H, D, —C₁-C₁₀ alkyl, —C₂-C₆ alkenyl, —C₂-C₆ alkynyl, —OC₁-C₆ alkyl, —O-cycloalkyl, aryl, C₁ alk-aryl, heteroaryl, cycloalkyl, cycloalkenyl, C₁ alk-heteroaryl, heterocycloalkyl, or heterocycloalkenyl;
each R^{d} is independently H, D, —C₁-C₁₀ alkyl, —C₂-C₆ alkenyl, —C₂-C₆ alkynyl, —OC₁-C₆alkyl, —O-cycloalkyl, aryl, C₁alk-aryl, heteroaryl, cycloalkyl, cycloalkenyl, C₁alk-heteroaryl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group;
R₇ is H, D, OR^{a}, C₁₋₄alkyl, wherein the C₁₋₄alkyl are optionally substituted with at least one of D, halogen, —OH, —CN, an amine, cycloalkyl, or heterocycloalkyl;
R₁₀ is H, D, —NR^{c}R^{d}, —NR^{a}R^{c}, C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl and heteroarylalkyl are optionally substituted by 1-6 R groups selected from H, D, halogen, —OH, —CN, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or R₁ and R₁₀ together with the atoms to which they are both attached form a heterocyclic group which are optionally substituted with D, halogen, —OH, —CN, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

2. The compound of claim 1, wherein n is 0, 1, 2, 3 or 4.

3-10. (canceled)

11. The compound of claim 1, wherein at least one R₁ is H.

12. The compound of claim 1, wherein at least one R₁ is —C₁-C₆ alkyl.

13. (canceled)

14. The compound of claim 1, wherein at least one R₁ is halogen.

15. (canceled)

16. The compound of claim 1, wherein R₂ is halogen.

17. (canceled)

18. The compound of claim 1, wherein R₂ is C₁-C₈ haloalkyl.

19. (canceled)

20. The compound of claim 1, wherein at least one R₃ is H.

21. The compound of claim 1, wherein each R₃ is H.

22. The compound of claim 1, wherein at least one R₃ is —C₁-C₆ alkyl.

23. The compound of claim 1, wherein each R₃ is —C₁-C₆ alkyl.

24-25. (canceled)

26. The compound of claim 1, wherein R₄ is H.

27. The compound of claim 1, wherein R₄ is —C₁-C₆ alkyl.

28. (canceled)

29. The compound of claim 1, that is a compound of formula II: 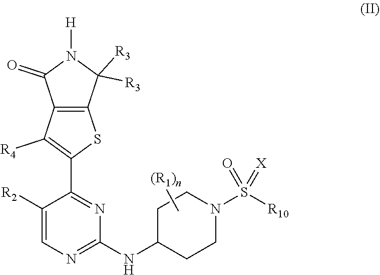
or a pharmaceutically acceptable salt or solvate or N-oxide thereof.

30. The compound of claim 1, that is a compound of formula III: 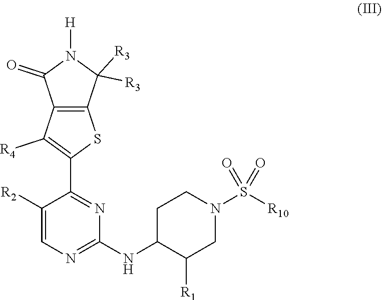
or a pharmaceutically acceptable salt or solvate or N-oxide thereof.

31. The compound of claim 1, that is a compound of formula IV: 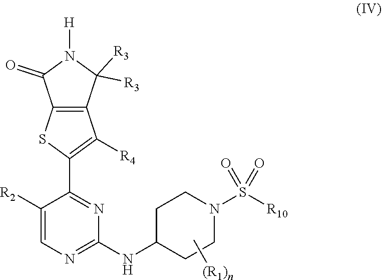
or a pharmaceutically acceptable salt or solvate or N-oxide thereof.

32. The compound of claim 1, that is a compound of formula V: 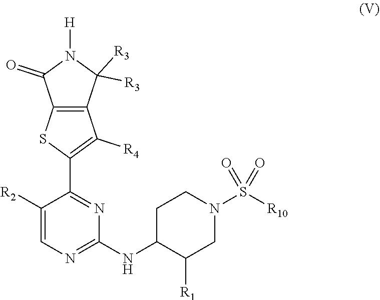
or a pharmaceutically acceptable salt or solvate or N-oxide thereof.

33. The compound of claim 1 that is:
2-(5-Fluoro-2-(((3R,4S)-3-methyl-1-(methylsulfonyl)piperidin-4-yl)amino)pyrimidin-4-yl)-3,6,6-trimethyl-5,6-dihydro-4H-thieno[2,3-c]pyrrol-4-one;
2-(5-fluoro-2-(((3R,4S)-3-fluoro-1-(methylsulfonyl)piperidin-4-yl)amino)pyrimidin-4-yl)-3,6,6-trimethyl-5,6-dihydro-4H-thieno[2,3-c]pyrrol-4-one;
2-(5-fluoro-2-((1-(methylsulfonyl)piperidin-4-yl)amino)pyrimidin-4-yl)-3,6,6-trimethyl-5,6-dihydro-4H-thieno[2,3-c]pyrrol-4-one;
2-(5-fluoro-2-((1-(methylsulfonyl)piperidin-4-yl)amino)pyrimidin-4-yl)-3-methyl-5,6-dihydro-4H-thieno[2,3-c]pyrrol-4-one;
2-(5-Fluoro-2-(((3R,4S)-3-methyl-1-(methylsulfonyl)piperidin-4-yl)amino)pyrimidin-4-yl)-3-methyl-5,6-dihydro-4H-thieno[2,3-c]pyrrol-4-one;
6,6-Dimethyl-2-(2-(((3R,4S)-3-methyl-1-(methylsulfonyl)piperidin-4-yl)amino)-5-(trifluoro-methyl)pyrimidin-4-yl)-5,6-dihydro-4H-thieno[2,3-c]pyrrol-4-one;
6,6-dimethyl-2-(2-(((3R,4S)-3-methyl-1-((1-methyl-1H-pyrazol-4-yl)sulfonyl)piperidin-4-yl)amino)-5-(trifluoromethyl)pyrimidin-4-yl)-5,6-dihydro-4H-thieno[2,3-c]pyrrol-4-one;
6,6-dimethyl-2-(2-(((3R,4S)-3-methyl-1-((1-methyl-1H-imidazol-4-yl)sulfonyl)piperidin-4-yl)amino)-5-(trifluoromethyl)pyrimidin-4-yl)-5,6-dihydro-4H-thieno[2,3-c]pyrrol-4-one;
2-(2-(((3R,4S)-1-((1H-pyrazol-4-yl)sulfonyl)-3-methylpiperidin-4-yl)amino)-5-(trifluoro-methyl)pyrimidin-4-yl)-6,6-dimethyl-5,6-dihydro-4H-thieno[2,3-c]pyrrol-4-one;
2-(2-(((3R,4S)-1-((1H-imidazol-4-yl)sulfonyl)-3-methylpiperidin-4-yl)amino)-5-(trifluoro-methyl)pyrimidin-4-yl)-6,6-dimethyl-5,6-dihydro-4H-thieno[2,3-c]pyrrol-4-one;
6,6-dimethyl-2-(2-((1-((1-methyl-1H-pyrazol-4-yl)sulfonyl)piperidin-4-yl)amino)-5-(trifluoromethyl)pyrimidin-4-yl)-5,6-dihydro-4H-thieno[2,3-c]pyrrol-4-one;
6,6-dimethyl-2-(2-((1-(methylsulfonyl)piperidin-4-yl)amino)-5-(trifluoromethyl) pyrimidin-4-yl)-5,6-dihydro-4H-thieno[2,3-c]pyrrol-4-one;
2-(2-(((3R,4R)-3-fluoro-1-(methylsulfonyl)piperidin-4-yl)amino)-5-(trifluoromethyl) pyrimidin-4-yl)-6,6-dimethyl-5,6-dihydro-4H-thieno[2,3-c]pyrrol-4-one;
2-(2-(((3R,4S)-3-fluoro-1-(methylsulfonyl)piperidin-4-yl)amino)-5-(trifluoromethyl) pyrimidin-4-yl)-6,6-dimethyl-5,6-dihydro-4H-thieno[2,3-c]pyrrol-4-one;
2-(2-(((3R,4S)-3-fluoro-1-((1-methyl-1H-imidazol-4-yl)sulfonyl)piperidin-4-yl)amino)-5-(trifluoromethyl)pyrimidin-4-yl)-6,6-dimethyl-5,6-dihydro-4H-thieno[2,3-c]pyrrol-4-one;
2-(2-(((3R,4R)-3-fluoro-1-((1-methyl-1H-pyrazol-4-yl)sulfonyl)piperidin-4-yl)amino)-5-(trifluoromethyl)pyrimidin-4-yl)-6,6-dimethyl-5,6-dihydro-4H-thieno[2,3-c]pyrrol-4-one;
2-(2-((1-(methylsulfonyl)piperidin-4-yl)amino)-5-(trifluoromethyl)pyrimidin-4-yl)-5,6-dihydro-4H-thieno[2,3-c]pyrrol-4-one;
2-(2-(((3R,4S)-3-methyl-1-(methylsulfonyl)piperidin-4-yl)amino)-5-(trifluoromethyl) pyrimidin-4-yl)-5,6-dihydro-4H-thieno[2,3-c]pyrrol-4-one;
2-(2-(((3R,4S)-3-fluoro-1-(methylsulfonyl)piperidin-4-yl)amino)-5-(trifluoromethyl) pyrimidin-4-yl)-5,6-dihydro-4H-thieno[2,3-c]pyrrol-4-one;
2-(2-((1-((1-methyl-1H-pyrazol-4-yl)sulfonyl)piperidin-4-yl)amino)-5-(trifluoromethyl) pyrimidin-4-yl)-5,6-dihydro-4H-thieno[2,3-c]pyrrol-4-one;
2-(2-(((3R,4S)-3-methyl-1-((1-methyl-1H-pyrazol-4-yl)sulfonyl)piperidin-4-yl)amino)-5-(trifluoromethyl)pyrimidin-4-yl)-5,6-dihydro-4H-thieno[2,3-c]pyrrol-4-one;
2-(2-(((3R,4S)-1-((1H-pyrazol-4-yl)sulfonyl)-3-fluoropiperidin-4-yl)amino)-5-(trifluoro-methyl)pyrimidin-4-yl)-5,6-dihydro-4H-thieno[2,3-c]pyrrol-4-one;
2-(2-((1-((1-Methyl-1H-pyrazol-4-yl)sulfonyl)piperidin-4-yl)amino)-5-(trifluoromethyl) pyrimidin-4-yl)-4,5-dihydro-6H-thieno[2,3-c]pyrrol-6-one;
2-(2-((1-(Methylsulfonyl)piperidin-4-yl)amino)-5-(trifluoromethyl)pyrimidin-4-yl)-4,5-dihydro-6H-thieno[2,3-c]pyrrol-6-one;
or a pharmaceutically acceptable salt thereof.

34. A pharmaceutical composition comprising a compound according to claim 1, or a pharmaceutically acceptable salt thereof, and a pharmaceutically acceptable excipient.

35. A method of inhibiting CDK2 and CDK4 and CDK6 comprising contacting the CDK2 and CDK4 and CDK6 with a compound according claim 1, or a pharmaceutically acceptable salt thereof, or a pharmaceutical composition comprising the compound.

36. A method for treating a disorder mediated by CDK2 and CDK4 and CDK6 in a patient in need thereof, comprising administering to said patient a compound according to claim 1, or a pharmaceutically acceptable salt thereof, or a pharmaceutical composition comprising the compound.

37. The method according to claim 36, wherein the disorder is a cancer.

38. The method according to claim 37, wherein the cancer is breast cancer, malignant brain tumors, colon cancer, small-cell lung cancer, non-small-cell lung cancer, bladder cancer, ovarian cancer, prostate cancer, chronic lymphoid leukemia, lymphoma, myeloma, acute myeloid leukemia, secondary pancreatic cancer or secondary brain metastases.

39. The method according to claim 38, wherein the breast cancer is HR+/HER2− or HR+/HER2+ advanced or metastatic breast cancer; and the malignant brain tumors are glioblastoma, astrocytoma, or pontine glioma.

40. The method according to claim 36, wherein the patient is administered a pharmaceutical composition comprising the compound.

41. The method according to claim 36, wherein the administration is oral administration.

42. The method according to claim 36, further comprising administering an additional therapeutic agent to the patient.

43. The method according to claim 42, wherein the additional therapeutic agent is a PRMT5 inhibitor, a HER2 kinase inhibitor, an aromatase inhibitor, an estrogen receptor antagonist or an alkylating agent.

44. The method according to claim 43, wherein the aromatase inhibitor is letrozole: the estrogen receptor antagonist is fulvestrant; and the alkylating agent is temozolomide.

45-46. (canceled)
